(19) United States
(12) Patent Application Publication
(10) Pub. No.: US 2023/0054563 A1
(43) Pub. Date: Feb. 23, 2023

(54) COMPOSITION AND METHODS OF MANUFACTURE

(71) Applicant: Phoenix Eagle Company Pty Ltd, Subiaco (AU)

(72) Inventors: Mark RICHARDSON, Subiaco (AU); Keith BROCKLEHURST, (US); Giuseppe TRIGIANTE, London (GB)

(21) Appl. No.: 17/788,986

(22) PCT Filed: Dec. 22, 2020
(86) PCT No.: PCT/IB2020/062349

§ 371 Date: Jun. 24, 2022

(30) Foreign Application Priority Data
Dec. 27, 2019 (GB) 1919385.3

(51) Int. Cl. C12N 9/50; A61K 8/66; A61Q 19/00; A61K 36/185; A61P 17/02

(52) U.S. Cl. CPC C12N 9/63; A61K 8/66; A61Q 19/00; A61K 36/185; A61P 17/02; A61K 38/00

(57) ABSTRACT

The present invention relates to compositions comprising Carica papaya derived serine protease, methods of extracting the protease from fruit sources as well as cosmetic and therapeutic uses thereof, and associated kits.

### FIELD OF THE INVENTION

The present invention relates to compositions comprising at least one protease identified in the Carica papaya plant, methods for producing the compositions from the Carica papaya plant, compositions comprising the at least one protease obtained or obtainable by the aforementioned methods, the use of such compositions in the manufacture of medicaments and cosmetics, the use of such compositions in the treatment of diseases and disorders including wounds, the use of such compositions in cosmetic applications, and associated kits for carrying out a method or use of the invention.

### BACKGROUND TO THE INVENTION

The effective management of many types of chronic wounds including but not limited to diabetic foot ulcers, pressure ulcers, venous ulcers and arterial ulcers remains a challenge. Such wounds may require a significant amount of time to heal, or never truly heal, and require significant resources from medical professionals. Numerous products have previously been developed to assist wound healing, such as a dressing of D-glucose polysaccharide obtained by hydrolysis of starch, containing ascorbic acid, collagen type I and alpha-tocopherol acetate (U.S. Pat. No. 6,187,743).

There has also been previous interest in the use of proteases such as papain and bromelain for wound debridement.

### SUMMARY OF THE INVENTION

We have identified in a filtrate obtained by treating the flesh of ripe papaya (Carica papaya) with an alkali, the presence of a protease not previously identified as an active protease in that fruit. We have confirmed that this protease is a serine protease and that the filtrate contains both serine protease and cysteine protease activity. A serine protease identified in secretions from medicinal maggots (van der Plas et al., 2014, PLoS ONE 9(3): e92096) has previously been shown to enhance plasminogen activator-induced fibrinolysis by degrading plasminogen and it was suggested that this may contribute to the ability of medicinal maggots to debride wounds of necrotic tissue.

Accordingly, in a first aspect, the present invention provides a composition comprising an active serine protease extracted from the ripe fruit of the Carica papaya plant, such as an active serine protease extracted from alkali-treated, pulped ripe Carica papaya fruit.

In one embodiment, the active serine protease is substantially free of insoluble Carica papaya-derived material.

In a second embodiment, the serine protease comprises a contiguous amino acid sequence having at least 95, 96, 97, 98 or 99% homology to (such as sequence identity with) amino acids 113 to 771 of SEQ ID NO:1. In a particular embodiment the serine protease has a molecular weight of from 65 to 75 kDa, such as about 70 kDa.

In a third embodiment the serine protease has a molecular weight of from 45 to 55 kDa and comprises a contiguous amino acid sequence having at least 95% homology to amino acids 142 to 618 of SEQ ID NO:1. Thus, the present invention provides a composition comprising an isolated, active serine protease having a molecular weight of from 45 to 55 kDa and comprising a contiguous amino acid sequence having at least 95% homology to amino acids 142 to 618 of SEQ ID NO:1.

The present invention also provides a composition comprising an isolated, active serine protease substantially free of insoluble Carica papaya-derived material, wherein the serine protease has a molecular weight of from 45 to 55 kDa and comprises a contiguous amino acid sequence having at least 95% homology to amino acids 142 to 618 of SEQ ID NO:1.

In a second aspect, the present invention provides a composition comprising a mixture of one or more active serine proteases derived or derivable from Carica papaya and one or more active cysteine proteases derived or derivable from Carica papaya. In one embodiment, the one or more active proteases are extracted from ripe Carica papaya. In another embodiment, the one or more active proteases are produced using a recombinant expression system.

The present invention also relates to methods of preparing compositions comprising at least one active serine protease by extracting the protease from ripe Carica papaya. Accordingly, in a third aspect, the present invention provides a process for preparing a composition comprising an active serine protease, which method comprises treating pulped ripe Carica papaya fruit with an alkali without subjecting the pulp to a heating step. In one embodiment, the alkali is a weak alkali, preferably sodium bicarbonate.

The compositions made by this process may further comprise one or more cysteine protease derived or derivable from Carica papaya.

In one embodiment, at least one soluble protease is separated from insoluble plant material after the alkali treatment.

The composition may be further treated to increase the concentration of the protease or proteases, for example, using freeze drying, dialysis, size exclusion chromatography or a combination thereof.

In a fourth aspect, the present invention further provides a composition comprising a serine protease obtained or obtainable by the methods of the invention.

The compositions may be formulated to be better suited to pharmaceutical or cosmetic applications. Accordingly, in a fifth aspect, the present invention provides a pharmaceutical composition comprising an active Carica papaya serine protease together with a pharmaceutically acceptable carrier or diluent, as well as a cosmetic composition comprising an active Carica papaya serine protease together with a cosmetically acceptable carrier or diluent. In a related aspect the present invention provides the composition of any of the first, second or fourth aspects of the invention together with a pharmaceutically acceptable carrier or diluent, as well as a cosmetic composition comprising a composition of any of the first, second or fourth aspects of the invention together with a cosmetically acceptable carrier or diluent.

The compositions of the invention may be used in a range of pharmaceutical and cosmetic applications. Accordingly, in a sixth aspect, the present invention also provides a method of treating a patient suffering from a skin condition, the method comprising administering a composition of the invention to the affected area of the patient. In some embodiments, the composition is used for the treatment of diseases and disorders including wounds. In some embodiments, the composition is used for debridement. In some embodiments, the composition is used for treating burns. In some embodiments, the composition is used for treating ulcers. In some embodiments, the composition is used for treating gangrene. In some embodiments, the composition is applied topically.

The present invention further provides in a seventh aspect a method of achieving a desired cosmetic outcome, such as smoothing and renewal of the epidermis of an individual, the method comprising applying a composition of the invention to the affected area of the patient. The present invention also provides a cosmetic method using the compositions of the invention. In some embodiments, the composition is used for exfoliation, lightening skin, or for applying to wrinkles, skin blemishes, freckles, pimples, acne, rosacea, sun spots, scars or varicose veins, or for applying to dry, aged or damaged skin.

In an eighth aspect, the present invention provides use of the compositions of the first, second, fourth or fifth aspects in the manufacture of a medicament. In some embodiments, the medicament is for the treatment of diseases and disorders including wounds. In some embodiments, the medicament is for debridement. In some embodiments, the medicament is for treating burns. In some embodiments, the medicament is for treating ulcers. In some embodiments, the medicament is for treating gangrene.

In a ninth aspect, the present invention provides use of the compositions of the first, second, fourth or fifth aspects in the manufacture of a cosmetic. In some embodiments, the cosmetic is for exfoliation.

In a tenth aspect, the present invention provides the compositions of the first, second, fourth or fifth aspects, for use in the treatment of diseases and disorders including wounds. In some embodiments, the treatment is debridement. In some embodiments, the treatment is for ulcers. In some embodiments, the composition is applied topically.

In an eleventh aspect, the present invention provides the compositions of the first, second, fourth or fifth aspects, for use in exfoliation.

In a twelfth aspect, the present invention provides kits comprising the compositions of the first, second, fourth or fifth aspects. In one embodiment, the kits are used for performing the methods of the present invention.

The compositions of the present invention can be formulated, for example, as capsules, tablets, creams, ointments, solutions, pastes, drops, sprays, aerosols, vapours, wipes, patches, gauzes, gels or liquids. Accordingly, in one embodiment, the compositions of the present invention are provided or packaged as capsules, tablets, creams, ointments, solutions, pastes, drops, sprays, aerosols, vapours, wipes, patches, gauzes, gels or liquids and do not need to be reconstituted prior to use.

### Definitions

Unless defined otherwise, all technical and scientific terms used herein have the same meaning as commonly understood by those of ordinary skill in the art to which the invention belongs. Although any methods and materials similar or equivalent to those described herein may be used in the practice or testing of the present invention, preferred methods and materials are described. For the purposes of the present invention, the following terms are defined below.

By “about” is meant a quantity, level, value, number, frequency, percentage, dimension, size, amount, weight or length that varies by as much 20, 15, 10, 9, 8, 7, 6, 5, 4, 3, 2 or 1% to a reference quantity, level, value, number, frequency, percentage, dimension, size, amount, weight or length.

By “active protease” is meant a protease that is proteolytically active.

Throughout this specification, unless the context requires otherwise, the words “comprise,” “comprises” and “comprising” will be understood to mean the inclusion of a stated step or element or group of steps or elements but not the exclusion of any other step or element or group of steps or elements. Thus, use of the term “comprising” and the like indicates that the listed steps or elements are required or mandatory, but that other steps or elements are optional and may or may not be present.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “debridement” refers to the removal of dead and damaged tissue from a wound.

The term “derivable” may be used interchangeably with the term “obtainable”.

The term “derived” may be used interchangeably with the term “obtained”.

By “isolated” is meant material that is substantially or essentially free from some or all of the components that normally accompany it in its native state. For example, an “isolated protease,” as used herein, refers to in vitro isolation and/or partial purification of a peptide or polypeptide protease molecule from its natural cellular environment, and from association with some or all of the components of the cell.

The term “obtainable”, when used in relation to proteins or compositions of the present invention, includes proteins or compositions produced not only by a particular specified method, but also the same proteins or compositions however produced, for example, by sourcing proteases from fruits or vegetables, or by recombinant DNA technology or other genetic engineering methods, for example, by using a recombinant expression system.

The terms “patient”, “subject” and “individual” are used interchangeably and refer to patients, subjects and individuals of human or other animals and include any one for whom it is desired to a treat, prevent, ameliorate, or reduce the severity of a disease, disorder or condition using the invention. However, it will be understood that “patient” does not imply that symptoms are present. Suitable animals, such as mammals, that fall within the scope of the invention include, but are not restricted to, primates (e.g. humans, chimpanzees) livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes).

The terms “wild-type” and “naturally occurring” are used interchangeably to refer to a protein that has the characteristics of that protein when isolated from a naturally occurring source. A wild type protein (e.g., a polypeptide) is that which is most frequently observed in a population and is thus arbitrarily designated the “normal” or “wild-type” form of the protein.

The term “wound” means an injury to living tissue in which the skin is cut or broken and includes dermal ulcers and burns. Dermal ulcers may include diabetic ulcers, pressure ulcers, venous (or varicose) ulcers and arterial ulcers.

The reference to any prior art in this specification is not, and should not be taken as an acknowledgement or any form of suggestion that prior art forms part of the common general knowledge of the person skilled in the art.

The entire content of all publications, patents, patent applications and other material recited in this specification is incorporated herein by reference.

### DETAILED DESCRIPTION OF THE INVENTION

Compositions Comprising Serine Proteases

The present invention relates to compositions comprising at least one active serine protease that can be extracted from or otherwise derived from the ripe fruit of the Carica papaya plant, such as following treatment of the pulped ripe fruit of the Carica papaya plant with an alkali, such as sodium bicarbonate.

It is to be understood that the term “active serine proeteases” refers to one or to more than one active serine protease.

In one embodiment, the active serine protease has an amino acid sequence comprising one or more of the sequences shown in SEQ ID NOs: 2 to 5, such as SEQ ID NOs: 2, 3 and 4, or all four sequences shown in SEQ ID NOs: 2, 3, 4 and 5, respectively.

The at least one active serine protease typically comprises a contiguous sequence having at least 95% sequence homology (preferably sequence identity), such as 96, 97, 98 or 99% sequence homology (preferably sequence identity) with amino acids 142 to 618 of SEQ ID NO:1, which is the catalytic domain of the protein containing the key catalytic triad residues Asp, His and Ser (shown in SEQ ID NO: 1 at residues 151, 221 and 558 respectively) and the conserved substrate-binding site Asn at residue 326.

SEQ ID NO: 1 (His 221 and peptides of SEQ ID NOs: 2 to 19 shown in bold)

| | |
|---|---|
| MAVSNPTLYL LSFLLFSISL TPVIASKSSY VVYLGAHSHG LELSSADLDR | 50 |
| VKESHYDFLG SFLGSPEEAQ ESIFYSYTKH INGFAAELND EVAAKLAKHP | 100 |
| KVVSVFLNKG RKLHTTRSWD FLGLEQNGVV PSSSIWKKAR FGEDTIIGNL | 150 |
| DTGVWPESKS FSDEGLGPIP SKWRGICDHG KDSSFHCNRK LIGARFFNRG | 200 |
| YASAVGSLNS SFESPRDNEG HGTHTLSTAG GNMVANASVF GLGKGTAKGG | 250 |
| SPRARVAAYK VCWPPVLGNE CFDADILAAF DAAIHDRVDV LSVSLGGTAG | 300 |
| GFFNDSVAIG SFHAVKHGIV VVCSAGNSGP DDGSVSNVAP WQITVGASTM | 350 |
| DREFPSYVLL GNNMSFKGES LSDAVLPGTN FFPLISALNA KATNASNEEA | 400 |
| ILCEAGALDP KKVKGKILVC LRGLNARVDK GQQAALAGAV GMILANSELN | 450 |
| GNEIIADAHV LPASHISFTD GLSVFEYINL TNSPVAYMTR PKTKLPTKPA | 500 |
| PVMAAFSSKG PNIVTPEILK PDITAPGVNV IAAYTRAQGP TNQNFDRRRV | 550 |
| QFNSVSGTSM SCPHVSGIVG LLKTLYPSWS PAAIRSAIMT SATTMDNINE | 600 |
| SILNASNVKA TPFSYGAGHV QPNQAMNPGL VYDLNTKDYL KFLCALGYSK | 650 |
| TLISIFSNDK FNCPRTNISL ADFNYPSITV PELKGLITLS RKVKNVGSPT | 700 |
| TYRVTVQKPK GISVTVKPKI LKFKKAGEEK SFTVTLKMKA KNPTKEYVFG | 750 |
| ELVWSDEDEH YVRSPIVVKA A | 771 |

Amino acids 1-25 are a putative signal sequence and amino acids 26 to 112 are a putative pro region that is cleaved to form the mature protein.

SEQ ID NO 2 to SEQ ID NO 5 are peptide fragments of SEQ ID NO 7. SEQ ID NO 12, SEQ ID NO 14 and SEQ ID NO 15 respectively.

SEQ ID NO: 2
SFSDEGLGPI PSK
SEQ ID NO: 3
GESLSDAVLP GTNFFPLISA LNAK
SEQ ID NO: 4
GPNIVTPEIL KPDITAPGVN VIAAYTR
SEQ ID NO: 5
TLYPSWSPAA IR
SEQ ID NO: 6
SWDFLGLEQN GVVPSSSIWK
SEQ ID NO: 7
FGEDTIIGNL DTGVWPESKS FSDEGLGPIP SK
SEQ ID NO: 8
GICDHGKDSS FHCNR
SEQ ID NO: 9
GARFFNRGYA SAVGSLNSSF ESPR
SEQ ID NO: 10
VCWPPVLGNE CFDADILAAF DAAIHDR
SEQ ID NO: 11
HGIVVVCSAG NSGPDDGSVS NVAPWQITVG ASTMDR
SEQ ID NO: 12
FKGESLSDAV LPGTNFFPLI SALNAKATNA SNEEAILCEA GALDPK
SEQ ID NO: 13
ILVCLR
SEQ ID NO: 14
TLPTKPAPVM AAFSSKGPNI VTPEILKPDIT APGVNVIAA
YTRAQGPTNQ NFDR
SEQ ID NO: 15
VQFNSVSGTS MSCPHVSGIV GLLKTLYPSWS PAAIR
SEQ ID NO: 16
ATPFSYGAGH VQPNQAMNPG LVYDLNTK
SEQ ID NO: 17
TLISIFSNDK FNCPRTNISL ADFNYPSITV PELK
SEQ ID NO: 18
GISVTVKPK
SEQ ID NO: 19
EYVFGELVWS DEDEHYVR

In one embodiment, the at least one active serine protease of the invention comprises the amino acid sequence from amino acids 118 to 763 of SEQ ID NO:1, with up to 1, 2, 3, 4, 5, 6, 7, 8, 9 or 10 or more, such as up to 3 or 5, amino acid modifications (deletions, insertions and/or substitutions) provided the protein retains serine protease activity.

A polypeptide may be modified in various ways including amino acid substitutions, deletions, truncations, and insertions. Methods for such manipulations are generally known in the art.

The serine proteases of the invention are in their active form i.e. not as inactive zymogens.

In one embodiment, the experimental results herein suggest that a protein isolated from Carica papaya and having an apparent molecular weight of 50 kDa was present as an active serine protease. Accordingly, in one embodiment at least one active serine protease of the invention may have a molecular weight of from 45 to 55 kDa, such as from 48 to 52 kDa, for example about 50 kDa. The molecular weight of the at least one protease of the invention may be about 45, 46, 47, 48, 49, 50, 51, 52, 53, 54 or 55 kDa. The molecular weight may be assessed by SDS PAGE, thus the protease has an average molecular weight as measured by SDS PAGE of from 45 to 55 kDa, such as from 48 to 52 kDa, for example about 50 kDa. Alternatively, the molecular weight can be calculated on the basis of the primary amino acid sequence.

The active serine protease(s) is(are) typically in substantially isolated form. This means that they have been at least partially purified from their Carica papaya source material or recombinantly produced. In one embodiment the isolated active serine proteases are substantially free of insoluble plant materials such as cellulose, lignin and the like. This will typically be as a result of one or more purification steps to remove insoluble materials leaving the soluble active serine proteases in solution. This will be described further below. “Substantially free of insoluble plant materials” means that less than 2% w/w, such as less than 1% w/w or 0.5% w/w, of the composition contains insoluble plant materials derived from the source material for the protease.

Compositions Comprising Mixtures of Serine Proteases and Cysteine Proteases

Cysteine proteases, also known as thiol proteases and cysteine endopeptidases (EC 3.4.22), are enzymes that degrade proteins. They share a common catalytic mechanism that involves a nucleophilic cysteine thiol in a catalytic triad or dyad. They occur in a variety of organisms. In particular, they are commonly found in fruits including papaya (Carica papaya and Vasconcellea cundianmarcensus), pineapple (Ananas comosus), fig (Ficus carica) and kiwifruit (Actinidia chinensis). In one embodiment, the cysteine protease is derived from a fruit. Thus, cysteine proteases include papain (EC 3.4.22.2), chymopapain (EC 3.4.22.6), bromelain (stem bromelain—EC 3.4.22.32 and fruit bromelain—EC 3.4.22.33), ficain (EC 3.4.22.3) and actinidain (EC 3.4.22.14).

It is to be understood that the term “active cysteine proteases” refers to one or to more than one active cysteine protease.

One or more active serine proteases derived from Carica papaya (by extraction or recombinantly produced) may be present in a mixture containing one or more active cysteine proteases, such as one or more of the cysteine proteases referred to above that are derived from Carica papaya. The process of the invention described further below results in one such mixture.

Thus, one source of the active cysteine proteases can be the same as for the active serine proteases, i.e. the fruit of the Carica papaya plant. Preferably the papaya is ripe. One advantage of ripe papaya as the source material instead of unripe papaya (which is the current commercial source of papain) is that the resulting composition comprising the cysteine protease has much lower levels of latex than typical commercial sources of papain. Latex can cause allergic reactions.

The active cysteine proteases are typically in substantially isolated form as described above for the active serine proteases. This means that they have been at least partially purified from its biological source material such as Carica papaya or recombinantly produced. In one embodiment the isolated cysteine proteases are substantially free of insoluble plant materials such as cellulose, lignin and the like. This will typically be as a result of one or more purification steps to remove insoluble materials to leave the soluble active protease in solution.

Processes for Obtaining Serine Proteases and Cysteine Proteases

A process of the invention can be used to isolate at least one active serine protease of the invention from ripe papaya fruit. Ripe papaya fruit is typically yellow over most of the exterior of the fruit. Unripe papaya (from the skin of which latex is harvested to produce papain) is green. Ripe papaya should not be hard when pressed but should give slightly and retain slight indentations. If it is very soft when pressed then it is overripe. In one embodiment papaya flesh is separated from the skin and seeds. In another embodiment the entire fruit is used including the skin and seeds. The fruit, such as the flesh, is then mechanically disrupted to form a pulp (for example in a blender until a smooth pulp is obtained).

The resulting pulp is then treated with an alkali. Suitable alkalis include strong bases such as NaOH and weak bases such as sodium bicarbonate. The alkali can be added as dry powder or in aqueous solution. In one embodiment the alkali is sodium bicarbonate and is typically added as a powder in an amount of at least 3% w/w, such as at least 5% w/w. The amount is typically less than 15% w/w, such as less than 12% w/w. In a particular embodiment the amount added is from 5 to 12% w/w.

In one embodiment, the alkali may have a pKa of less than 11. In some embodiments, the alkali may be a bicarbonate or a carbonate or a combination thereof. In some embodiments, the alkali may be a water-soluble alkali metal bicarbonate salt or a water-soluble alkali metal carbonate salt or a combination thereof. In some embodiments, the amount of alkali added may be from about 1% w/w to about 40% w/w, from about 1% w/w to about 35% w/w, from about 1% w/w to about 30% w/w, from about 1% w/w to about 25% w/w, from about 1% w/w to about 20% w/w, from about 1% w/w to about 15% w/w, from about 1% w/w to about 10% w/w, from about 2% w/w to about 40% w/w, from about 2% w/w to about 35% w/w, from about 2% w/w to about 30% w/w, from about 2% w/w to about 25% w/w, from about 2% w/w to about 20% w/w, from about 2% w/w to about 15% w/w, from about 2% w/w to about 10% w/w, from about 3% w/w to about 40% w/w, from about 3% w/w to about 35% w/w, from about 3% w/w to about 30% w/w, from about 3% w/w to about 25% w/w, from about 3% w/w to about 20% w/w, from about 3% w/w to about 15% w/w, from about 3% w/w to about 10% w/w, from about 4% w/w to about 40% w/w, from about 4% w/w to about 35% w/w, from about 4% w/w to about 30% w/w, from about 4% w/w to about 25% w/w, from about 4% w/w to about 20% w/w, from about 4% w/w to about 15% w/w, from about 4% w/w to about 10% w/w, from about 5% w/w to about 40% w/w, from about 5% w/w to about 35% w/w, from about 5% w/w to about 30% w/w, from about 5% w/w to about 25% w/w, from about 5% w/w to about 20% w/w, from about 5% w/w to about 15% w/w or from about 5% w/w to about 10% w/w. In one embodiment, the amount of alkali added may be from about 1% w/w to about 20% w/w. In a preferred embodiment, the amount of alkali added may be from about 1% w/w to about 15% w/w. In a particularly preferred embodiment, the amount of alkali added may be from about 4% w/w to about 15% w/w.

In one embodiment the alkali is added in an amount such that the final pH of the composition after the alkali has reacted with the pulp is at least 7.5, such as from pH 7.5 to pH 11.5. In some embodiments, the composition may have a final pH in the range of from about 7.0 to about 14.0, from about 7.0 to about 13.5, from about 7.0 to about 13.0, from about 7.0 to about 12.5, from about 7.0 to about 12.0, from about 7.0 to about 11.5, from about 7.0 to about 11.0, from about 7.0 to about 10.5, from about 7.0 to about 10.0, from about 7.0 to about 9.5, from about 7.0 to about 9.0, from about 7.0 to about 8.5, from about 7.0 to about 8.0, from about 7.0 to about 7.5, from about 7.1 to about 14.0, from about 7.1 to about 13.5, from about 7.1 to about 13.0, from about 7.1 to about 12.5, from about 7.1 to about 12.0, from about 7.1 to about 11.5, from about 7.1 to about 11.0, from about 7.1 to about 10.5, from about 7.1 to about 10.0, from about 7.1 to about 9.5, from about 7.1 to about 9.0, from about 7.1 to about 8.5, from about 7.1 to about 8.0, from about 7.1 to about 7.5, from about 7.2 to about 14.0, from about 7.2 to about 13.5, from about 7.2 to about 13.0, from about 7.2 to about 12.5, from about 7.2 to about 12.0, from about 7.2 to about 11.5, from about 7.2 to about 11.0, from about 7.2 to about 10.5, from about 7.2 to about 10.0, from about 7.2 to about 9.5, from about 7.2 to about 9.0, from about 7.2 to about 8.5, from about 7.2 to about 8.0, from about 7.2 to about 7.5, from about 7.3 to about 14.0, from about 7.3 to about 13.5, from about 7.3 to about 13.0, from about 7.3 to about 12.5, from about 7.3 to about 12.0, from about 7.3 to about 11.5, from about 7.3 to about 11.0, from about 7.3 to about 10.5, from about 7.3 to about 10.0, from about 7.3 to about 9.5, from about 7.3 to about 9.0, from about 7.3 to about 8.5, from about 7.3 to about 8.0, from about 7.3 to about 7.5, from about 7.4 to about 14.0, from about 7.4 to about 13.5, from about 7.4 to about 13.0, from about 7.4 to about 12.5, from about 7.4 to about 12.0, from about 7.4 to about 11.5, from about 7.4 to about 11.0, from about 7.4 to about 10.5, from about 7.4 to about 10.0, from about 7.4 to about 9.5, from about 7.4 to about 9.0, from about 7.4 to about 8.5, from about 7.4 to about 8.0, from about 7.4 to about 7.5, from about 7.5 to about 14.0, from about 7.5 to about 13.5, from about 7.5 to about 13.0, from about 7.5 to about 12.5, from about 7.5 to about 12.0, from about 7.5 to about 11.5, from about 7.5 to about 11.0, from about 7.5 to about 10.5, from about 7.5 to about 10.0, from about 7.5 to about 9.5, from about 7.5 to about 9.0, from about 7.5 to about 8.5, or from about 7.5 to about 8.0. In a particularly preferred embodiment, the composition may have a final pH in the range of from about 7.5 to about 9.5.

The reaction is performed with the pulp at a temperature of 30° C. or less, preferably without subjecting the pulp to any heating step. The reaction may therefore for example be performed at room temperature, such as standard room temperature and pressure. It can also be carried out at lower temperatures, such as about 4° C. Accordingly, in some embodiments, the reaction is performed in a range of about 4° C. to about 25° C., or about 4° C. to about 22° C., or about 4° C. to about 20° C.

Once the alkali has had sufficient time to react with the pulp, the treated pulp is then subjected to a separation step to remove insoluble plant materials (in the case of sodium bicarbonate, the separation step can be performed once the treated pulp is no longer effervescing). For example, the treated pulp can be centrifuged and/or filtered. Centrifugation can be performed at for example between about 6000 and 18000 g. In one embodiment the treated pulp is centrifuged and the resulting supernatent is then filtered through a 0.22 μm filter.

The resulting composition can be subject to one or more purification steps to concentrate and/or further separate the proteases of the invention from other cellular components. Suitable techniques include (i) gel filtration (size exclusion) chromatography to separate the proteins specifically on the basis of molecular weight e.g. using Sephadex G-100 as described in the examples; (ii) ionic exchange chromatography; (iii) dialysis; (iv) ammonium acetate precipitation and/or (v) freeze-drying. Resulting compositions/fractions from chromatography can be tested to confirm the continued presence of protease activity using, for example, the assays described in the examples (L-BapNA, optionally with a serine protease inhibitor or a cysteine protease inhibitor added during the assay to determine that the protease activity is due to serine protease or cysteine protease activity). The known molecular weight of the at least one serine protease of the invention can also be used to confirm the presence and amount of the at least one serine protease e.g. by SDS-PAGE.

Purification processes may also conveniently be used to adjust the pH and/or salt concentration to levels suitable for the formulation of the composition into a pharmaceutical or cosmetic product.

In another embodiment, the process may further comprise a step of beating the mixture following addition of the alkali. In a further embodiment, the process may further comprise a step of filtering the mixture to obtain a filtrate. In a further embodiment, the process may further comprise a step of filtering the mixture to obtain a residue. In a further embodiment, the process may further comprise a step of freezing and thawing the composition. The mixture obtained may be frozen and thawed prior to filtering.

The final composition is preferably substantially free of insoluble plant materials, for example containing less than about 2%, 1%, 0.5% or 0.1% w/w insoluble material. In one embodiment, the percentage of protease by weight of total protein is in the range of about 0.01% to 5%. The final composition may comprise from about 1 to 100 IU/ml of serine protease activity. In some embodiments, final composition may comprise from about 1 to about 10 IU/ml, about 1 to about 20 IU/ml, about 1 to about 30 IU/ml, about 1 to about 40 IU/ml, about 1 to about 50 IU/ml, about 1 to about 60 IU/ml, about 1 to about 70 IU/ml, about 1 to about 80 IU/ml, about 1 to about 90 IU/ml, about 10 to about 100 IU/ml, about 20 to about 100 IU/ml, about 30 to about 100 IU/ml, about 40 to about 100 IU/ml, about 50 to about 100 IU/ml, about 60 to about 100 IU/ml, about 70 to about 100 IU/ml, about 80 to about 100 IU/ml, about 90 to about 100 IU/ml, about 1 to about 90 IU/ml, about 10 to about 90 IU/ml, about 20 to about 90 IU/ml, about 30 to about 90 IU/ml, about 40 to about 90 IU/ml, about 50 to about 90 IU/ml, about 60 to about 90 IU/ml, about 70 to about 90 IU/ml, about 80 to about 90 IU/ml, about 1 to about 80 IU/ml, about 10 to about 80 IU/ml, about 20 to about 80 IU/ml, about 30 to about 80 IU/ml, about 40 to about 80 IU/ml, about 50 to about 80 IU/ml, about 60 to about 80 IU/ml, about 70 to about 80 IU/ml, about 1 to about 70 IU/ml, about 10 to about 70 IU/ml, about 20 to about 70 IU/ml, about 30 to about 70 IU/ml, about 40 to about 70 IU/ml, about 50 to about 70 IU/ml, about 60 to about 70 IU/ml, about 1 to about 60 IU/ml, about 10 to about 60 IU/ml, about 20 to about 60 IU/ml, about 30 to about 60 IU/ml, about 40 to about 60 IU/ml, about 50 to about 60 IU/ml, about 1 to about 50 IU/ml, about 10 to about 50 IU/ml, about 20 to about 50 IU/ml, about 30 to about 50 IU/ml, about 40 to about 50 IU/ml, about 1 to about 40 IU/ml, about 10 to about 40 IU/ml, about 20 to about 40 IU/ml, about 30 to about 40 IU/ml, about 1 to about 30 IU/ml, about 10 to about 30 IU/ml, about 20 to about 30 IU/ml, about 1 to about 20 IU/ml, or about 10 to about 20 IU/ml of serine protease activity.

Formulation of Pharmaceutical Compositions

Compositions comprising at least one serine protease of the invention, and optionally cysteine proteases, such as proteases derived from or derivable from Carica papaya, can be combined with pharmaceutically acceptable carriers or diluents to form a pharmaceutical composition. As used herein, although a pharmaceutical composition of the invention will typically contain water, the term “pharmaceutically acceptable carriers or diluents” excludes water.

The carriers and diluents described in the following section for cosmetic compositions can typically also be used for pharmaceutical compositions. Such compositions can for example, be in the form of a cream; a lotion; a serum; an ointment; or a gel. The compositions can also be applied onto/impregnated into solid forms such as a wound dressing, for example a gauze pad and the like.

Formulation of Cosmetic Compositions

Compositions comprising the at least one serine protease of the invention, and optionally also comprising cysteine proteases, such as proteases derived from or derivable from Carica papaya, can also be combined with cosmetically acceptable carriers or diluents to form a cosmetic composition. As used herein, although a cosmetic composition of the invention will typically contain water, the term “cosmetically acceptable carriers or diluents” excludes water.

Cosmetic compositions according to the present invention can suitably include all active materials and appropriate components conventionally known for such formulations. Such components include, for instance, gelling agents, anionic polymers, thickeners, surfactants, hydrating agents, emollients, chelating agents, antioxidants, preservatives, buffering compounds, perfumes, fillers, colourings, volatile or non-volatile, modified or non-modified silicones and reducing agents. The relative proportions of the different components are suitably those that are used in conventional cosmetic formulations. It is envisaged that the person skilled in the art will naturally select the optionally present materials and incorporate these into the composition according to the invention in such a manner that the advantageous properties associated with the desirous enzyme activity are not significantly modified or eliminated. It is further envisaged that certain components may fulfil more than one criteria, for instance, glycerine is known to act as both a solvent and a humectant.

As mentioned above, a variety of components may be present in the compositions of the present invention; foremost of these components is likely to be water, as a solvent. Amounts of water may range from about 1% to about 95%, such as from about 25% to about 90%, by weight of the composition. Other suitable solvents include glycerine and benzyl alcohol.

The cosmetic compositions of the present invention may include one or more emulsifiers, for example, if the composition is in the form of a lotion, cream, gel or serum. Suitable emulsifiers that can be used in the present invention include, for example, one or more alkoxylated fatty alcohols, C₁₄₋₂₂ alcohols, alkylpolyglycosides, C₁₄₋₂₀ alkylglucoside, saponifiers, alkyl sulfates, monoalkyl and dialkyl phosphates, alkyl sulphonates, acyl isothionates, cetyl alcohol, stearyl alcohol, sorbitans, stearic acid, glyceryl stearate or any combinations thereof.

Certain oil-in-water emulsion-based compositions of the invention may also include one or more stabilisers to stabilise the emulsion. Suitable stabilisers include, for example, alcohols, alkoxylated alcohols, fatty alcohols, glyceryl esters, such as, glyceryl stearate, gums, soaps, synthetic polymers, waxes, or any combinations thereof. Particularly suitable emulsion stabilisers include a stearyl alcohol or glyceryl stearate.

The compositions of the invention may further include one or more emollients to soften and soothe the skin. Suitable emollients comprise, for example, one or more fats and oils (hydrogenated or non-hydrogenated), such as, vegetable oil, castor oil, coconut oil and cocoa butter, shea butter, as well as esters, such as, tricapryl citrate and isononyl isonanoate, or any combinations thereof.

The compositions of the invention may include one or more humectants. A humectant is a component that absorbs or retains moisture. Suitable humectants that can be used in the present composition include, for example, urea, pyroglutamic acid, amino acids (e.g. glutamic acid), polyols or other compounds with hygroscopic properties, or any combinations thereof. Typically, the humectant is a polyol, such as a sorbitol. In a composition of the invention in the form of a serum, the primary humectants present include methyl-gluceth-20 and propylene glycol.

Suitable preservatives for use in the compositions of the present invention include, for example, one or more alkanols such as phenoxy ethanol, ethylenediaminetetraacetic acid (EDTA) salts, EDTA fatty acid conjugates, isothiazolinone, parabens such as methylparaben and propylparaben, propylene glycols, sorbates, urea derivatives such as diazolidinyl urea and imidazolidinyl urea, quaternary ammonium salts, or any combinations thereof.

Suitable chelating agents for inclusion in the compositions of the invention may include, for example EDTA derivatives, or any combinations thereof.

The composition may include one or more buffering compounds that serve to adjust and maintain the pH of the composition. Suitable buffering agents include, for example, one or more adipic acids, glycines, citric acids, calcium hydroxides, magnesium aluminometa-silicates, triethanolamine, or any combinations thereof.

When present, the buffering agent should be present at a level suitable to ensure stable pH in the composition throughout its normal shelf life and period of use.

The cosmetic compositions may also include one or more surfactants. Surfactants are of particular importance in compositions of the invention that are for the purposes of skin washes, such as soaps or face washes. Suitable surfactants include, for example, anionic, non-ionic, cationic, amphoteric, or any combinations thereof. Preferably the one or more surfactants are non-ionic surfactants. Suitable nonionic surfactants include, for example, one or more alkoxylated alcohols, ethoxylated alcohols, propoxylated alcohols, inter-dispersed ethoxylated-propoxylated alcohols, copolymers, fatty acids, alkyl phenols, polyglycosides, polyglucosides, n-alkylpyrrolidones, block copolymers, or any combinations thereof.

Typically, the cosmetic compositions of the invention are in the form of a cream; a lotion; a serum; a face wash; a shampoo; a foam; a skin wash; a skin tonic; an ointment; or a gel. However, the skilled person will appreciate that any composition suitable for administering the active enzymes of the invention to the epidermis can be used.

It is envisaged that the compositions of the invention should contain sufficient proteolytic activity to enable from about 0.1 IU/ml to about 100 IU/ml of total serine protease activity to be applied to the skin per dose, wherein a dose is an amount of between about 2 ml and about 10 ml of product. In some embodiments, the compositions of the invention should contain sufficient proteolytic activity to enable from about 1 to about 10 IU/ml, about 1 to about 20 IU/ml, about 1 to about 30 IU/ml, about 1 to about 40 IU/ml, about 1 to about 50 IU/ml, about 1 to about 60 IU/ml, about 1 to about 70 IU/ml, about 1 to about 80 IU/ml, about 1 to about 90 IU/ml, about 10 to about 100 IU/ml, about 20 to about 100 IU/ml, about 30 to about 100 IU/ml, about 40 to about 100 IU/ml, about 50 to about 100 IU/ml, about 60 to about 100 IU/ml, about 70 to about 100 IU/ml, about 80 to about 100 IU/ml, about 90 to about 100 IU/ml, about 1 to about 90 IU/ml, about 10 to about 90 IU/ml, about 20 to about 90 IU/ml, about 30 to about 90 IU/ml, about 40 to about 90 IU/ml, about 50 to about 90 IU/ml, about 60 to about 90 IU/ml, about 70 to about 90 IU/ml, about 80 to about 90 IU/ml, about 1 to about 80 IU/ml, about 10 to about 80 IU/ml, about 20 to about 80 IU/ml, about 30 to about 80 IU/ml, about 40 to about 80 IU/ml, about 50 to about 80 IU/ml, about 60 to about 80 IU/ml, about 70 to about 80 IU/ml, about 1 to about 70 IU/ml, about 10 to about 70 IU/ml, about 20 to about 70 IU/ml, about 30 to about 70 IU/ml, about 40 to about 70 IU/ml, about 50 to about 70 IU/ml, about 60 to about 70 IU/ml, about 1 to about 60 IU/ml, about 10 to about 60 IU/ml, about 20 to about 60 IU/ml, about 30 to about 60 IU/ml, about 40 to about 60 IU/ml, about 50 to about 60 IU/ml, about 1 to about 50 IU/ml, about 10 to about 50 IU/ml, about 20 to about 50 IU/ml, about 30 to about 50 IU/ml, about 40 to about 50 IU/ml, about 1 to about 40 IU/ml, about 10 to about 40 IU/ml, about 20 to about 40 IU/ml, about 30 to about 40 IU/ml, about 1 to about 30 IU/ml, about 10 to about 30 IU/ml, about 20 to about 30 IU/ml, about 1 to about 20 IU/ml, or about 10 to about 20 IU/ml of total serine protease activity to be applied to the skin per dose, wherein a dose is an amount of between about 2 ml and about 3 ml, about 2 ml and about 4 ml, about 2 ml and about 5 ml, about 2 ml and about 6 ml, about 2 ml and about 7 ml, about 2 ml and about 8 ml, about 2 ml and about 9 ml, about 3 ml and about 4 ml, about 3 ml and about 5 ml, about 3 ml and about 6 ml, about 3 ml and about 7 ml, about 3 ml and about 8 ml, about 3 ml and about 9 ml, about 3 ml and about 10 ml, about 4 ml and about 5 ml, about 4 ml and about 6 ml, about 4 ml and about 7 ml, about 4 ml and about 8 ml, about 4 ml and about 9 ml, about 4 ml and about 10 ml, about 5 ml and about 6 ml, about 5 ml and about 7 ml, about 5 ml and about 8 ml, about 5 ml and about 9 ml, about 5 ml and about 10 ml, about 6 ml and about 7 ml, about 6 ml and about 8 ml, about 6 ml and about 9 ml, about 6 ml and about 10 ml, about 7 ml and about 8 ml, about 7 ml and about 9 ml, about 7 ml and about 10 ml, about 8 ml and about 9 ml, about 8 ml and about 10 ml, or about 9 ml and about 10 ml of product.

Serine protease activity can be measured by the L-BApNA assay described in the examples with the use of a serine protease inhibitor during the assay to determine the serine protease component. Alternatively, the benzoyl-L-arginine ethyl ester (BAEE) assay described in GB 2,440,117 may be used. In one embodiment the serine protease activity is in the range of from about 5 units to about 60 units per application dose.

The compositions of the invention may also contain a similar level of cysteine protease activity. Cysteine protease activity can be measured by the L-BApNA assay described in the examples with the use of a cysteine protease inhibitor during the assay to determine the cysteine protease component.

A combination of a serine protease from Aspergillus melleus and a cysteine protease in the form of a cosmetic serum has previously been shown in a volunteer group of 40 women to improve over a period of 14-28 days skin smoothness and decrease wrinkles, as determined using measurements of skin surface micro topography (GB 2,440,117). The compositions of the present invention can also be used in various cosmetic applications such as a cosmetic method for promoting smoothing and renewal of the epidermis of an individual.

Uses of Compositions

Usually, the cosmetic compositions of the invention are applied to the skin of the face, hands and neck, however other skin surfaces may be subjected to the cosmetic treatment. The only contraindication is that particularly sensitive areas of skin, such as inflamed skin or the skin close to the eyes be avoided. The compositions of the invention are usually applied directly to the skin surface manually by the user as is conventional.

The methods of the invention therefore include applying a cosmetic composition of the invention to the epidermis of the individual.

The pharmaceutical compositions of the present invention can be used in a variety of pharmaceutical applications related to the treatment of diseases, disorders and conditions, including skin conditions and wounds. The compositions of the present invention can also be used in a variety of cosmetic applications.

Accordingly, the present invention further provides methods of debridement of wounds comprising the topical application of a composition of the invention, compositions of the present invention for use in a method of treating wounds, methods of treating an individual suffering from burns, wherein the method comprises administering topically, or by other routes of administration, to an affected area of the individual a preparation of the invention, compositions of the invention for use or when used in a method of treating wounds, methods of enhancing wound healing, comprising administering topically, or by other routes of administration, to a wound a composition of the invention, methods of exfoliating or lightening skin comprising applying to the skin a cosmetic composition of the invention, uses of a cosmetic composition of the invention to exfoliate or lighten skin, methods of treating dry, aged or damaged skin comprising applying to the skin a cosmetic composition of the invention, and uses of a cosmetic composition of the invention to treat dry, aged or damaged skin.

The protease compositions of the present invention may in particular be used to prevent, treat, reduce or ameliorate a variety of skin conditions including wounds, including chronic wounds such as diabetic ulcers, pressure ulcers, venous ulcers and arterial ulcers, and other skin conditions including but not limited to, eczema, psoriasis, acne, rosacea, ichthyosis, vitiligo, hives, seborrheic dermatitis.

In one preferred embodiment, the pharmaceutical compositions can be used to debride wounds. Such wounds will typically contain necrotic tissue and include diabetic ulcers, pressure ulcers, venous ulcers, arterial ulcers and the like. Thus such wounds may be chronic wounds. In use, the compositions (or dressings etc. that are impregnated with a composition) are applied to the affected area. This will typically be carried out at least once daily, such as twice daily. The amount of composition and frequency of application can be determined by a physician.

Compositions of the present invention may be administered therapeutically or cosmetically. In such applications, compositions may be administered to a subject already suffering from a condition, in an amount sufficient to cure or at least partially arrest the condition and any complications. The quantity of the composition should be sufficient to effectively treat the patient.

The compositions may also be administered in the form of liposomes. Liposomes may be derived from phospholipids or other lipid substances, and may be formed by mono- or multi-lamellar hydrated liquid crystals dispersed in an aqueous medium. Any non-toxic, physiologically acceptable and metabolisable lipid capable of forming liposomes may be used. The compositions in liposome form may contain stabilisers, preservatives and excipients. Preferred lipids include phospholipids and phosphatidyl cholines (lecithins), both natural and synthetic. Methods for producing liposomes are known in the art, and in this regard specific reference is made to: Prescott, Ed., Methods in Cell Biology, Volume XIV, Academic Press, New York, N.Y. (1976), p. 33 et seq., the contents of which are incorporated herein by reference.

The composition of the present invention may be used in the manufacture of a medicament for preventing, treating, reducing or ameliorating a skin condition of a subject.

The composition of the present invention may be also used in the manufacture of a cosmetic for preventing, treating, reducing or ameliorating a skin condition of a subject.

Dosages

The “therapeutically effective” dose level for any particular patient will depend upon a variety of factors including the condition being treated and the severity of the condition, the activity of the composition employed, the age, body weight, general health, sex and diet of the patient, the time of administration, the route of administration, the duration of the treatment, and any drugs used in combination or coincidental with the treatment, together with other related factors well known in the art. One skilled in the art would therefore be able, by routine experimentation, to determine an effective, non-toxic amount of the composition which would be required to treat applicable conditions.

Further, it will be apparent to one of ordinary skill in the art that the optimal quantity and spacing of individual dosages of the composition will be determined by the nature and extent of the condition being treated, the form, route and site of administration, and the nature of the particular individual being treated. Also, such optimum conditions can be determined by conventional techniques.

It will also be apparent to one of ordinary skill in the art that the optimal course of treatment, such as the number of doses of the composition given per day for a defined number of days, can be ascertained by those skilled in the art using conventional course of treatment determination tests.

Routes of Administration

The compositions of the present invention can be administered by standard routes. In general, the compositions may be administered by topical routes. Typically, compositions of the present invention are administered topically to the affected area of an individual.

In other embodiments, the compositions may be administered by other enteral/enteric routes, such as rectal, sublingual or sublabial, or via the central nervous system, such as through epidural, intracerebral or intracerebroventricular routes. Other locations for administration may include via epicutaneous, transdermal, intradermal, nasal, intraarterial, intracardiac, intraosseus, intrathecal, intraperitoneal, intravesical, intravitreal, intracavernous, intravaginal or intrauterine routes.

Timing of Therapies

Typically, in therapeutic applications, the treatment would be for the duration of the disease state.

Those skilled in the art will appreciate that the compositions disclosed herein may be administered as a single agent or as part of a combination therapy approach to the methods disclosed herein, either at diagnosis or subsequently thereafter, for example, as follow-up treatment or consolidation therapy as a compliment to currently available therapies for such treatments. The compositions disclosed herein may also be used as preventative therapies for subjects who are genetically or environmentally predisposed to developing such diseases.

The compositions may be administered regularly for as long as is needed, such as until an improvement in the condition is seen. Therefore they may be administered hourly, multiple times a day, daily, multiple times a week, weekly, monthly or at whatever frequency is deemed appropriate.

Kits

Kits of the present invention facilitate the employment of the methods and uses of the present invention. Typically, kits for carrying out a method or use of the invention contain all the necessary reagents and means to carry out the method. For example, in one embodiment, the kit may comprise a composition of the present invention and, optionally, means to administer the composition such as devices for point of care methods.

Typically, the kits described herein will also comprise one or more containers. In the context of the present invention, a compartmentalised kit includes any kit in which compositions are contained in separate containers, and may include small glass containers, plastic containers or strips of plastic or paper. Such containers may allow the efficient transfer of compositions from one compartment to another compartment whilst avoiding cross-contamination of compositions, and the addition of agents or solutions of each container from one compartment to another in a quantitative fashion.

Typically, a kit of the present invention will also include instructions for using the kit to perform the appropriate methods and uses.

Methods, uses, compositions and kits of the present invention are equally applicable to any animal, including humans, for example including non-human primate, equine, bovine, ovine, caprine, leporine, avian, feline and canine species. Accordingly, for application to different species, a single kit of the invention may be applicable, or alternatively different kits, for example containing compositions specific for each individual species, may be required.

The person skilled in the art will understand and appreciate that different features disclosed herein may be combined to form combinations of features that are within the scope of the present invention.

The present invention will now be described further with reference to the following examples, which are illustrative and non-limiting. The examples refer to the following figures.

### DESCRIPTION OF THE FIGURES

FIG. 1: Overlay-zymography schematic representation. Overlay-zymography process and subsequent excision of bands from enzyme activity spot to process on SDS PAGE gels.

FIG. 2: Representation of debriding assay using Franz cell diffusion system (Shi, Ermis et al. 2009).

FIG. 3: Bar chart plot of the L-BApNA proteolytic activity of OPAL A and OPAL B filtrates. OPAL A and OPAL B filtrates on L-BApNA, as measured by the slopes of the time-course. OPAL B filtrate has almost 8-fold more catalytic activity as compared to OPAL A filtrate. Results are represented as the mean±S.D for n=8 experiments. Structural analysis was carried out using student T-test (*** p<0.001).

FIG. 4: Spectrophotometric plot of enzymatic hydrolysis of L-BApNA by OPAL B filtrate and with addition of E-64. Representative plot showing enzymatic hydrolysis of the substrate L-BAPNA by OPAL B filtrate, and partial activity inhibition by cysteine protease inhibitor E-64, suggesting the presence of other proteases in addition to cysteine proteases.

FIG. 5: Spectrophotometric plot of the effect of protease inhibitor cocktail (PIC) on the proteolytic activity of OPAL B filtrate. Representative trace showing enzymatic hydrolysis of the substrate L-BAPNA by OPAL B filtrate, and its complete inhibition by the PIC.

FIG. 6: Bar chart plot of effect of different protease inhibitor classes on the proteolytic activity of OPAL B filtrate.

FIG. 7: Spectrophotometric plot of the effect of E-64+AEBSF on the proteolytic activity of OPAL B filtrate. Representative trace showing the complete inhibition of L-BAPNA activity in OPAL B filtrate by the combination of cysteine protease inhibitor E-64 and serine protease inhibitor AEBSF.

FIG. 8: Overlay zymography analysis of OPAL B filtrate.

FIG. 9: Overlay zymography analysis of OPAL B filtrate and effect on using inhibitors. Native PAGE gel bands in OPAL B samples were obtained and the protein bands were subsequently transferred from gel to nitrocellulose membrane. Zymography analysis shows the two enzymatic activities in control and complete inhibition of upper band ( - - - ) with cysteine protease inhibitor E-64 and inhibition of lower band ( - - - ) with serine protease inhibitor AEBSF.

FIG. 10: Silver staining of SDS PAGE gel run with elutions 2-10. The high intensity band present in the 6^{th} fraction at 80 kDa as well as bands at 50 k Da and 30 kDa, were analyzed using LC/MS analysis and revealed as a subtilase.

FIG. 11: SDS PAGE western blotting analysis of OPAL B filtrate treated with serine protease probe using various treatments. SDS PAGE western blotting analysis of OPAL B filtrate treated with 1 μl probe (per 100 μl) with no tris-HCl (lane 2) and with 5 mg/ml tris-HCl (lane 4), 10 mg/ml tris-HCL (lane 6) and respectively with AEBSF (lanes 3, 5 and 7). OPAL B filtrates with 1 μL probe and 10-mg/mL tris-HCl with E-64 (lane 6) and with E-64 and AEBSF (lane 7) showing no background disturbance.

FIG. 12: Differential digestions of AWE substrate (percentage activity) by OPAL A filtrate as compared to latex-papain, showing OPAL A is better at digesting collagen and fibrin whereas control latex papain is better at digesting elastin. Results are represented as the mean±SD for n=3 experiments. Structural analysis was carried out using student T-test (** p<0.01, *** p<0.001).

FIG. 13: Differential digestion of AWE substrate (percentage activity) by dialyzed OPAL A and OPAL A+10% urea as compared to native OPAL A filtrate. Dialyzed OPAL A had enhanced debriding activity on both fibrin and elastin but not collagen which correlates with the lower activity of OPAL A towards collagen. OPAL A addition of urea inhibited the activity of enzymes towards Fibrin and Elastin in the AWE assay. Results are represented as the mean±S.D for n=3 experiments. Structural analysis was carried out using student T-test (** p<0.01, *** p<0.001).

FIG. 14: In vivo case study regarding management of severe pressure ulcer with OPAL B, pre-commencement of treatment with Opal B. Central area of necrosis. Failed attempt to reduce the size of the ulcer with a romboid flap. Distal part of the flap dusky. Warning—graphic images.

FIG. 15: In vivo case study regarding management of severe pressure ulcer with OPAL B, treatment day 2. Delineation of area that will become necrotic from that which will be viable. Wound edge pink and better blood flow to the skin flap. Warning—graphic images.

FIG. 16: In vivo case study regarding management of severe pressure ulcer with OPAL B, treatment day 4. Clear demarcation between viable and non-viable tissue. Viable tissue looks clean and pink. Skin flap looks fully viable. Warning—graphic images.

FIG. 17: In vivo case study regarding management of severe pressure ulcer with OPAL B, treatment day 16. Wound debrided surgically, sutures removed and flap excised. Early granulation of the clean wound surface is evident. Wound edge is pink and suggestion of new skin growth (exposure adjusted to match skin colour of previous images). Warning—graphic images.

FIG. 18: In vivo case study regarding management of severe pressure ulcer with OPAL B, treatment day 19. Skin and wound colour becoming dusky, particularly wound edges. Wound remains clean (exposure adjusted to match skin colour of earlier images). Warning—graphic images.

### EXAMPLES

Preparation of OPAL A and OPAL B

The manufacturing process of OPAL A filtrate was performed in sterile conditions. The ends of the ripe fruit of the Carica papaya plant were trimmed and the skin was peeled. The flesh of the fruit was quartered and seeds were then excised. The papaya pieces were mixed in a blender until a smooth pulp was obtained. The pulp was placed in a beaker, which was inside a water bath set at 80° C. and stirred continuously until the temperature of the pulp was 55° C. (measured using a glass thermometer). 10% by weight dry sodium bicarbonate was added to the beaker. The beaker was then placed in the water bath, set at 55° C. and stirred slowly for 5 minutes. After the pulp containing sodium bicarbonate was centrifuged at 12000×g for 30 minutes, the supernatant was collected and transferred into sterile universal tubes by filtering using 0.22 μm filters (FDR-050-071N-Fisher Scientific). OPAL B filtrates were prepared with a one step change in the manufacturing process in that the heating step was omitted.

Gel Filtration Chromatography Using Sephadex G-100 Superfine

Gel filtration chromatography was used to separate the proteins specifically on the basis of molecular weight. 14 mL length chromatography columns (C3919-1EA-sigma Aldrich) with Sephadex G-100 (G10050-Sigma) (separation between 4 kDa-150 kDa) were packed and washed with 100 mM Tris-HCl with 0.15M NaCl at pH 8 overnight. 1 mL of the required samples was passed through the column separately in order to separate the proteins on the basis of molecular weight. The required number of 0.5 mL fractions were collected and A280 was measured. 50 μL of each fraction was then plated in 96 well plates and 50 μL of 1.5 mM L-BApNA buffer (made with 10 mM Tris-HCl pH 8.5 buffer) was added. L-BApNA activity at A410 was then measured both immediately and then after 2 hours using a Synergy HT microplate reader (cat. 12926527, Bio-Tek Instruments, Thermo Fisher Scientific, UK). The difference in values was plotted and the fractions with higher L-BApNA activity were run through SDS PAGE gels, which were then stained using Coomassie or silver staining.

L-BApNA (N-benzoyl-L-arginine 4-nitroanilide hydrochloride) Assays

A 1.5 mM stock solution of L-BApNA (B-3133 sigma) was prepared by dissolving 63 mg of L-BApNA in 1.5 ml dimethyl sulfoxide (DMSO), and then made up to 100 ml with water. Hydrolysis of the L-BApNA at the bond between the arginine and the p-nitroaniline moieties releases the chromophore p-nitroaniline, which can be detected by spectroscopy at an absorbance of 410 nm in International Units (IU). IU is defined as the amount of enzyme that causes an increase in absorbance of 0.01 units/min under excess substrate conditions (zero order kinetics). Solids specific activity is expressed in IU/mg, while in the case of liquid formulations, the activity is expressed in IU/mL.

To determine the optimal substrate concentration to maintain zero order kinetics throughout experiments, a series of titration runs were carried out with a range of substrate concentrations. The minimum concentration thus found is 0.2 mM or 1:6 dilution of the stock 1.5 mM solution. Table 1 shows reagent doses used for all kinetic experiments. Phosphate buffer at pH 6 was used in the L-BApNA assays because it gave the highest enzymatic activity reading compared to other buffers.

TABLE 1
L-BApNA assay final reagent concentrations

| Contents | Reference | Sample | |
|---|---|---|---|
| pH 6.0 Buffer | 333 | 333 | |
| Water | 501 | 333 | |
| Substrate (L-BApNA) | 166 | 166 | Zero at 410 nm |
| Enzyme (OPAL A/B) | 0 | 166 | Record A₄₁₀ |

To verify substrate purity, a controlled total hydrolysis catalyzed by alkali was carried out under the following conditions.

TABLE 2
L-BApNA substrate purity assay compositions

| | Reference cell (μl) | Sample Cell (μl) | |
|---|---|---|---|
| 5M NaOH | 0 | 966 | |
| Water | 966 | 0 | Zero at 410 nm |
| Substrate | 33 | 33 | Record A₄₁₀ |

$T hus [ subtrate ] = \frac{( {A}_{410} \times 30 ( dilution factor ) )}{8800 ( \begin{matrix}molar coefficient of extinction of p - nitroanaline \\ at 410 nm\end{matrix} )}$

Results confirmed 99% purity of the substrate as stated by manufacturer.

Inhibitors

E-64 (IU PAC name: (1S,2S)-2-(((S)-1-((4-Guanidinobutyl)amino)-4-methyl-1-oxopentan-2-yl) carbamoyl) cyclopropane carboxylic acid) is a strong and irreversible specific inhibitor of cysteine proteases. The trans-epoxysuccinyl group (active moiety) of E-64 irreversibly binds to the active thiol group of cysteine proteases, thereby inhibiting them. E-64 does not react with non-protease enzymes and does not inhibit serine proteases.

2,2′-dipyridyldisulfide (2DPS) is used as a thiol-specific reversible inhibitor for cysteine proteases, including papain, bromelain, and ficin.

Protease inhibitor cocktails (PICs) were formulated as shown in Table 3:

TABLE 3
The components of protease inhibitors in protease inhibitor cocktails

| | Protease inhibitor | Enzymes targeted |
|---|---|---|
| Reagent | (stock concentration) | by inhibitor |
| Protease Inhibitor Cocktail | E-64 (1.4 mM) | Cysteine Proteases |
| (P2714, Sigma) | Leupeptin (2 mM) | |
| | AEBSF (104 mM) | Serine Proteases |
| | Aprotinin (80 μM) | |
| | Leupeptin (2 mM) | |
| | Pepstatin A (1.5 mM) | Acid Proteases |
| | Bestatin (4 mM) | Amino peptidases |

Overlay Zymography

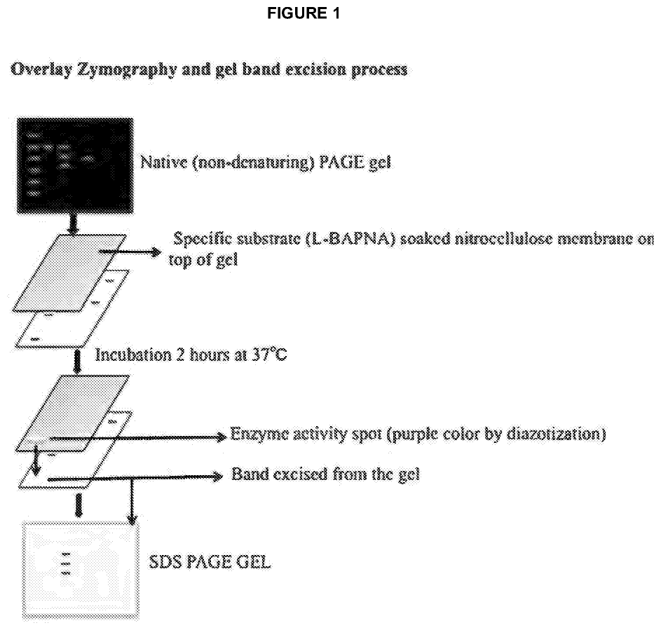

Overlay-zymography was carried out according to the procedure in Vinokurov et al. (2005) with some modifications (FIG. 1). Briefly, a nitrocellulose membrane was soaked in a 1.2 mg/mL L-BApNA solution in water. The membrane was subsequently left to air dry for 5 minutes and then laid on top of the native PAGE gel after running the samples through, which were slightly soaked in their respective running buffers. Two additional experiments were carried out in which membranes were soaked in 35M R-alanine, 0.14 M acetic acid, pH 4.3, with either 10 mM DTT or 25 mM cysteine. All zymographies were then incubated in a closed chamber at 37° C. for 30-60 min. The membrane was removed and again left to dry for 5 minutes and the p-nitroaniline was visualized by diazotization.

Diazotization was performed by following the protocol detailed in Hosseininaveh et al. (2009). Briefly, the membrane was soaked sequentially for 5 minutes in a sodium nitrite solution (1 mg/mL in 1 M HCl), then an ammonium sulfamate solution (5 mg/mL in 1M HCl) and finally a NNED solution (N-(1-naphthyl)-ethylenediamine dihydrochloride) (0.5 mg/mL in 48% v/v ethanol/water), for about 30 seconds to 1 minute until any diazotized p-nitroaniline became clearly visible as a purple smear/bands.

Mass Spectrometry

Protein bands identified with colloidal-coomassie staining were analyzed by MALDI-Mass fingerprinting at the PNAC Facility, Department of Biochemistry at the University of Cambridge. The gel bands were excised and subjected to the following treatment—30 min per step, 20° C., in 200 μL 100 mM ammonium bicarbonate/50% acetonitrile: 1) Reduction with 5 mM Tris (2-carboxyethyl) phosphine; 2) Alkylation by addition of iodoacetamide (25 mM final concentration); and 3) Removal of liquid and then wash.

Once washed, the gel pieces were dried in vacuo for 10 min and then 25 μl 100 mM ammonium bicarbonate containing 5 μg/mL modified trypsin (Promega) was added. Digestion was performed for 17 h at 32° C. Peptides were recovered and desalted using μC18 ZipTip (Millipore) and eluted to a maldi target plate using 1-2 μL alpha-cyano-4-hydroxycinnamic acid matrix (Sigma) in 50% acetonitrile/0.1% trifluoroacetic acid. Peptide masses were determined using a Bruker ultrafleXtreme Maldi mass spectrometer in reflectron mode and ms/ms fragmentation peroformed in LIFT mode. Data analysis was undertaken with FlexAnalysis, BioTools and ProteinScape software (Bruker). Database searches of the combined mass fingerprint data were performed using Mascot (Matrix Science). Where required, additional manipulation was performed through Protein Prospector.

Debriding Assay

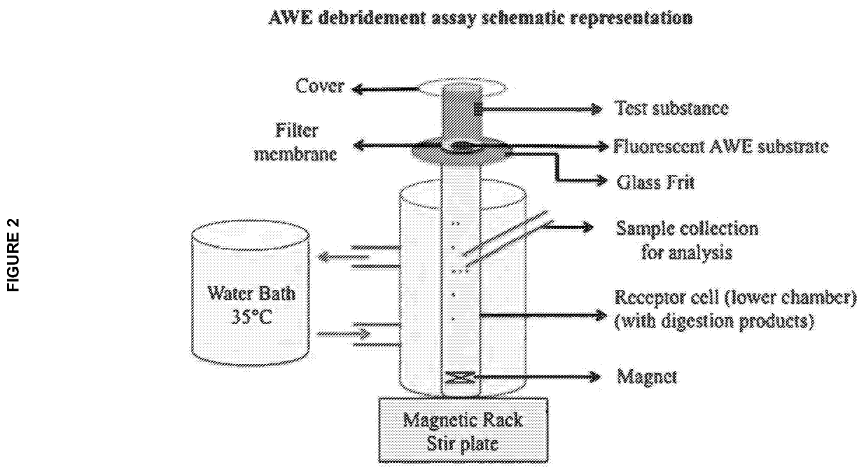

The AWE debriding assay is an in vitro surrogate of wound necrotic tissue proteolysis activity developed by Health Point (Shi, Ermis et al. 2009). It has been shown to compare well to in vivo animal data. The AWE substrate consists of a pellet of three wound related extra cellular matrix proteins (collagen, elastin and fibrin), each tagged with a different fluorophore. Gradual degradation of this matrix can be measured by progressive increase in fluorescence intensity in a Franz diffusion cell setup (FIG. 2). The final readings for the experiments are taken at 24 hours.

Materials for the debriding assay were: Collagen-Fluorescein Isothiocyanate (FITC) (CF308), Elastin-Rhodamine (R144) from Elastin products company, USA. Thrombin and fibrinogen (605157, 341573) from Merck chemicals limited. Tris Buffer containing 50 mM Tris with 100 mM NaCl and 10 mM CaCl₂, magnetic stirrer bar (Z328936), L-cysteine (W326305), Elastin-bovine (E1625), 7-Amino-4-Methyl Coumarin (A9891) and papain from papaya latex (P3375) all from Sigma.

Preparation of Fibrin-Coumarin: Fibrinogen was labelled with 7-amino-4-methyl coumarin by mixing fibrinogen in a coumarin solution (0.02 mg/mL in Tris buffer) with a final fibrinogen concentration of 10 mg/mL (in Tris buffer). The mixture was incubated at room temperature with rotary shaking for 1 hour. The coumarin-labelled fibrin was derived by adding a thrombin solution (2.5 units/mL) to the fibrinogen-coumarin solution and allowed to clot for 2 hours.

The formed clots were washed 3 times with water and allowed to stand overnight in distilled water and methanol (1:1) to remove excess dye. The clots were then transferred to a glass container with transparent non-sticky filter paper and allowed to dry for 3 days. The dried fibrin-coumarin was ground into fine powder using a mortar and pestle.

Preparation of Artificial Wound Eschar (AWE) Substrate

Collagen-FITC, elastin-rhodamine and fibrin-coumarin were mixed according to the composition referred to in Table 4. Except fibrinogen, all the materials were weighed into a 50 mL conical centrifuge tube. Using a tissue tearer the materials were homogenised in 10 mL Tris buffer for 3-5 minutes. A 10 mL, 15-mg/mL fibrinogen solution was prepared in Tris buffer pH 6.8 in a separate tube. The two solutions were combined and thoroughly mixed by using the tissue tearer for about 2 minutes. Thrombin solution (50 U/mL) was added and quickly mixed, and the solution was then poured into a petri dish containing a 90 mm non-reactive membrane and allowed to clot for 1 hour. The clotted substrate was then rinsed with water 3 times (5 minutes each) to remove thrombin. After washing, excess water was removed by using tissue paper and stored at 4° C. until further use.

Franz Diffusion Cell System

With the AWE substrate still attached to the membrane, a 9 mm diameter piece was punched out using a biopsy punch. The 9 mm AWE substrate was placed on the top of a non-reactive nitrocellulose membrane and placed in the Franz diffusion cell between the two chambers, of which the lower (receptor cell) was filled with Tris buffer containing 1% (v/v) penicillin-streptomycin (FIG. 2). The sample holder was placed on the top and the whole assembly was fastened with a screw clamp to the receptor cell and surrounded with a 35° C. water bath. The sample was then loaded in the upper chamber of the sample holder and covered with parafilm in which an air hole was made. The solution was mixed using a magnetic stirrer at 500 rpm throughout the experiment. At time zero and regular intervals up to 24 hours, 100 μL of solution was sampled from the receptor cell. The collected samples were loaded into a 96 well micro plate for immediate fluorescent measurement using a fluorescent plate reader (Synergy HT KC4 v3.4). After measurement, 100 μL of sample was replaced in the receptor cell.

The following equation was applied to determine the total cumulative digestion of all three-protein components in the substrate:

CDₙ=In×V_{cell}

Where n=time in hours, CDₙ and In are the cumulative digestion parameter and fluorescent intensity at hour n, and V_{cell} is the volume of the cell (5.1 mL).

TABLE 4
Composition of artificial wound Eschar (AWE) substrate

| | Wavelengths | |
|---|---|---|
| Component | excitation/emission | Percentage (%) |
| Collagen-FITC | 485 nm/520 nm | 65 |
| Fibrin-Coumarin | 365 nm/440 nm | 10 |
| Elastin-Rhodamine | 550 nm/570 nm | 10 |
| Fibrinogen | | 15 |

### Example 1—OPAL B Filtrate—a Variant of OPAL a Filtrate and its Proteolytic Activity

Carica papaya belongs to the small family of Caricaceae and is one of the major fruits cultivated in tropical and sub tropical zones for its edible fruit and latex. The fruits used in these examples are ripe papaya fruits, mainly obtained from Brazil and Jamaica, purchased in local UK-based supermarkets. Most of the well-characterized and intensively studied cysteine proteases are members of the papain family. Papain, chymopapain A and B, chymopapain M, and caricain have all been extracted from the latex of Carica papaya but have never been characterized from the ripe fruit.

We have previously carried out mass spectrometry work (Richard J. Lipscombe—Proteomics International laboratory, University of Western Australia) and showed that OPAL A filtrate from Carica papaya ripe fruit extract contains cysteine (thiol dependent) proteases. These cysteine proteases are traditionally extracted only from papaya latex or from the skin of papaya (Proteomics International, personal communication). To functionally identify cysteine proteases in OPAL A filtrates, spectrophotometric assays were performed. These experiments involved monitoring catalytic hydrolysis of the chromogenic substrate L-BApNA (Nα-benzoyl-L-arginine 4-nitroanilide hydrochloride) by the OPAL A filtrate.

Hydrolysis of L-BApNA in the presence of protease, at the bond between the arginine and p-nitroaniline moieties, releases the chromophore p-nitroaniline, which can then be detected by spectroscopy at a wavelength of 410 nm. The procedure for the L-BApNA activity determination is described in the methods above. Under the optimized conditions employed, the excess substrate yields zero order kinetics, which are apparent as a straight line, the slope of which is proportional to enzymatic activity.

We have previously established (unpublished data) that OPAL A has cysteine protease activity that is essentially abolished by the addition of the inhibitor E-64, that specifically inhibits cysteine proteases. We therefore previously concluded that the protease activity in OPAL A is essentially due only to the presence of active cysteine proteases. To determine the effect of omitting the heat treatment step, the protease activity in both OPAL A and OPAL B filtrates was tested using an L-BApNA assay. After 10 minutes, E-64 inhibitor was added.

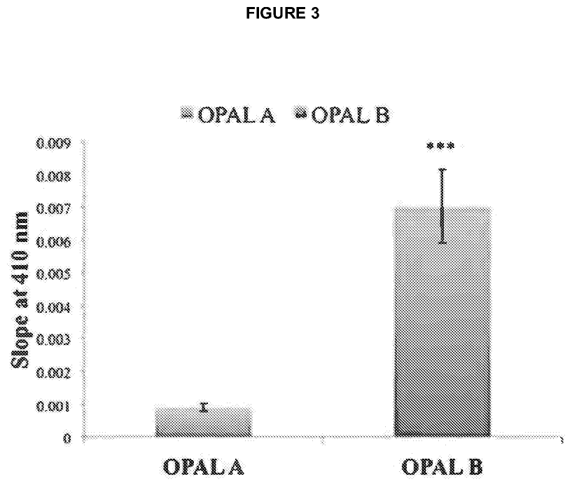

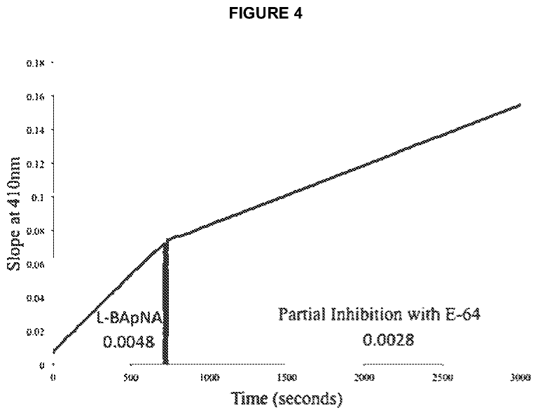

OPAL B appeared to exhibit an almost 8-fold increased catalytic activity (linear increase in A₄₁₀ substrate release over time), as compared to OPAL A (FIG. 3). Interestingly, addition of E-64 to OPAL B filtrate in excess did not result in complete inhibition of activity, as in OPAL A. Therefore, and without wishing to be bound by theory, the increase of A₄₁₀ values with substrate could be due to non-thiol-dependent (non-cysteine proteases) catalysis. The fact that treatment with the cysteine protease inhibitor E-64 did not completely abolish protease activity suggests that both cysteine and non-cysteine proteases were present in the OPAL B filtrate, as shown in FIG. 4.

### Example 2—Identification of Cysteine and Serine Proteases in OPAL B Filtrate Via Enzyme Kinetics

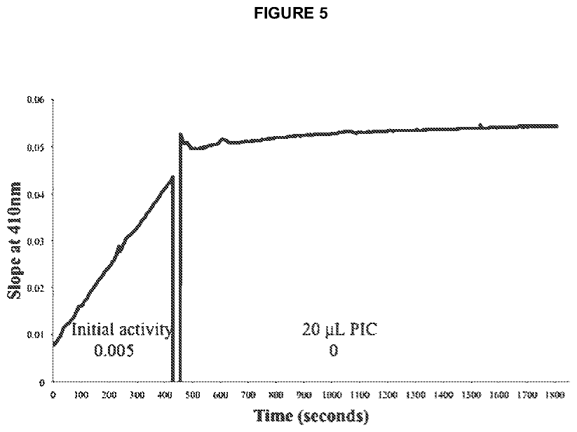

To better understand the non-thiol dependent or non-cysteine proteases in the OPAL B filtrate, enzymatic assays were carried out initially using a Protease Inhibitor Cocktail (PIC) (sigma P2714), containing all the essential protease inhibitors (see Table 3). Adding 20 μL of PIC to the OPAL B filtrate achieved complete inhibition of the protease activity, as shown in FIG. 5.

Based on these data, we next investigated the effect of each active inhibitor by individually testing each component of PIC that may be responsible for the enhanced activity of OPAL B. The components of the protease inhibitors present in the cocktail are shown in Table 3.

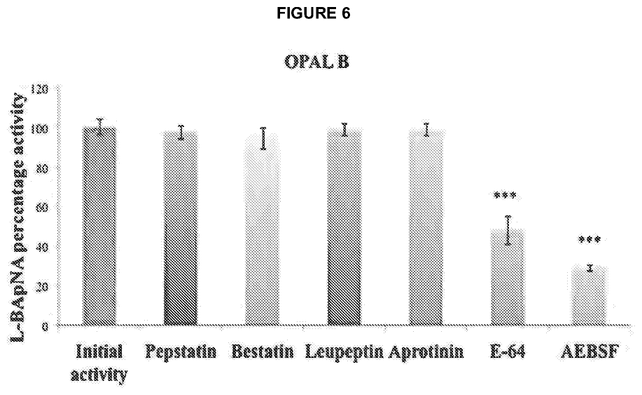

After testing each inhibitor present in the PIC with OPAL B filtrates, only two inhibitors were able to halt proteolytic activity. These were E-64 and AEBSF, as shown in FIG. 6, which are cysteine protease and serine protease inhibitors, respectively.

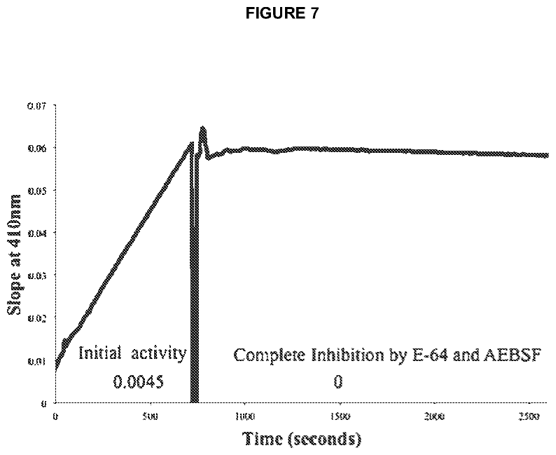

The combination of both inhibitors completely abrogates enzyme activity, as shown in FIG. 7. This suggests that the enzyme activity in OPAL B filtrate consists solely of cysteine and serine proteases.

### Example 3—Zymography Assays to Functionally Identify Proteases Determined by Enzyme Kinetics

OPAL B filtrate appears to be a complex mixture of many proteins. Therefore, to individually visualize the protease activities that were detected by spectrophotometry assays, we used a technique called overlay zymography. In this technique, proteins are first separated on a standard native PAGE gel, which preserves enzymatic activity. A membrane containing substrate solution (L-BApNA at 1.2 mg/mL) is then overlaid onto the gel, and developed using a colour-enhancing agent. This enables active proteases to be directly visualized on the membrane.

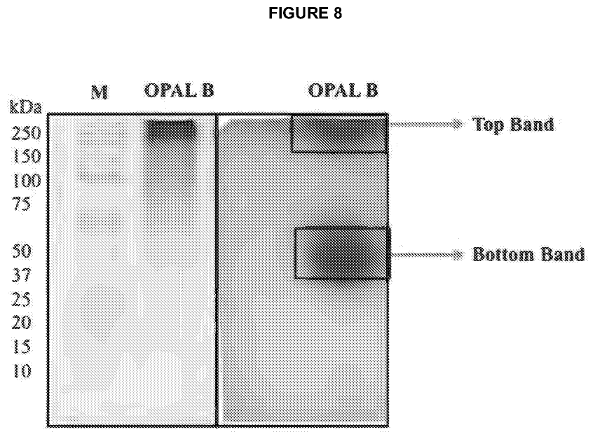

As shown in FIG. 8, Colloidal Blue stained native PAGE gel revealed bands in the OPAL B filtrate in unidirectional phase (anodic side). Transfer of protein from gel to nitrocellulose membrane and subsequent zymography analysis showed a stark increase in enzymatic activity (which shows two enzymatic activities).

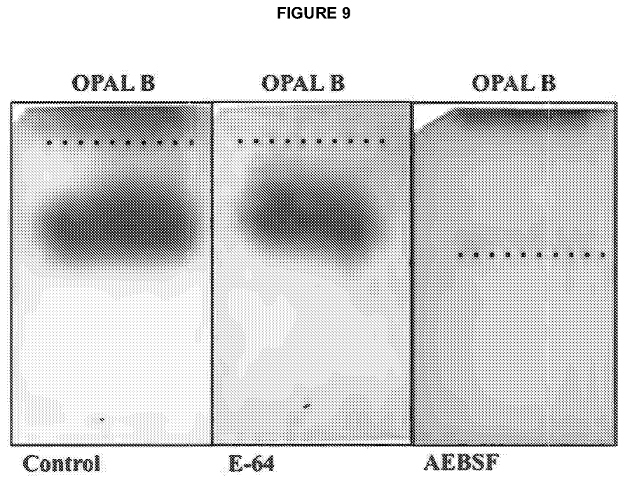

We further characterized the two-zymography bands obtained in FIG. 8 to verify that the observed two signals or bands are cysteine and serine proteases, respectively. The method was modified by including specific inhibitors (E-64 for cysteine proteases, and AEBSF for serine proteases), onto the nitrocellulose membrane before placing the membrane on top of the native PAGE gel. The data obtained clearly revealed that the bands are indeed cysteine protease(s) and serine protease(s) in the OPAL B concentrated filtrate. The cysteine protease(s) correspond to the upper band and the serine protease(s) correspond to the lower band, as shown in FIG. 9.

### Example 4—Size Exclusion Chromatography to Identify Serine Protease

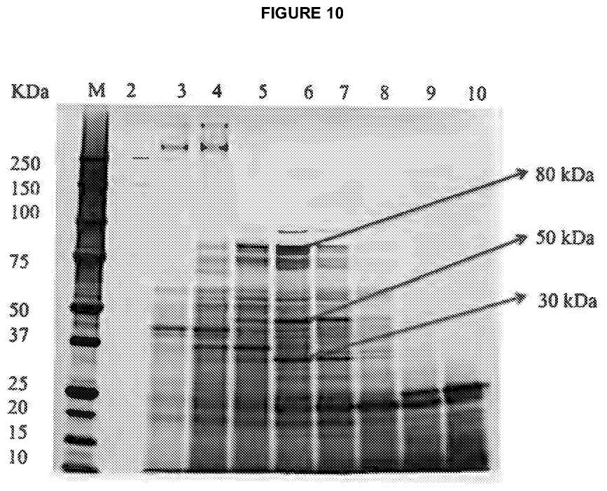

To purify serine protease(s), OPAL B was concentrated and purified through size exclusion chromatography using a sephadex G-100 column that separates proteins in the range of 4 kDa to 150 kDa. Dialysed OPAL B pre-treated with E-64 and with 100 mM L-Arginine (incubated for 2 hours) was run through a 14 mL sephadex G-100 column. Twenty-four elution fractions were collected and the 96 well plate L-BApNA assay was performed (data not shown). The fractions comprising the highest L-BApNA activity were selected for SDS PAGE gels, which were then silver stained, as shown in FIG. 10. The bands in the fractions, the abundance of which correlated with L-BApNA activity, were selected and analysed by LC/MS-MS analysis (namely the intensity bands present in the 6^{th} fraction at the 80 kDa, 50 kDa and 30 kDa bands). These were matched to a Carica papaya sequence shown in SEQ ID NO: 1 that had been putatively identified as a subtilase.

This result is consistent with an analysis of complete OPAL B filtrate which identified 4 fragments within SEQ ID NO: 1 as follows.

| MAVSNPTLYL LSFLLFSISL TPVIASKSSY VVYLGAHSHG LELSSADLDR | 50 |
|---|---|
| VKESHYDFLG SFLGSPEEAQ ESIFYSYTKH INGFAAELND EVAAKLAKHP | 100 |
| KVVSVFLNKG RK | |
| LHTTRSWD FLGLEQNGVV PSSSIWKKAR FGEDTIIGNL | 150 |
| DTGVWPESKS FSDEGLGPIP SKWRGICDHG KDSSFHCNRK LIGARFFNRG | 200 |
| YASAVGSLNS SFESPRDNEG HGTHTLSTAG GNMVANASVF GLGKGTAKGG | 250 |
| SPRARVAAYK VCWPPVLGNE CFDADILAAF DAAIHDRVDV LSVSLGGTAG | 300 |
| GFFNDSVAIG SFHAVKHGIV VVCSAGNSGP DDGSVSNVAP WQITVGASTM | 350 |
| DREFPSYVLL GNNMSFKGES LSDAVLPGTN FFPLISALNA KATNASNEEA | 400 |
| ILCEAGALDP KKVKGKILVC LRGLNARVDK GQQAALAGAV GMILANSELN | 450 |
| GNEIIADAHV LPASHISFTD GLSVFEYINL TNSPVAYMTR PKTKLPTKPA | 500 |
| PVMAAFSSKG PNIVTPEILK PDITAPGVNV IAAYTRAQGP TNQNFDRRRV | 550 |
| QFNSVSGTSM SCPHVSGIVG LLKTLYPSWS PAAIRSAIMT SATTMDNINE | 600 |
| SILNASNVKA TPFSYGAGHV QPNQAMNPGL VYDLNTKDYL KFLCALGYSK | 650 |
| TLISIFSNDK FNCPRTNISL ADFNYPSITV PELKGLITLS RKVKNVGSPT | 750 |
| TYRVTVQKPK GISVTVKPKI LKFKKAGEEK SFTVTLKMKA KNPTKEYVFG | 750 |
| SEQ ID NO: 1 | |
| ELVWSDEDEH YVRSPIVVKA A | 771 |
| SEQ ID NO: 2 | |
| SFSDEGLGPI PSK | |
| SEQ ID NO: 3 | |
| GESLSDAVLP GTNFFPLISA LNAK | |
| SEQ ID NO: 4 | |
| GPNIVTPEIL KPDITAPGVN VIAAYTR | |
| SEQ ID NO: 5 | |
| TLYPSWSPAA IR | |

### Example 5—Reactivity Probe to Identify Active Form of Serine Protease

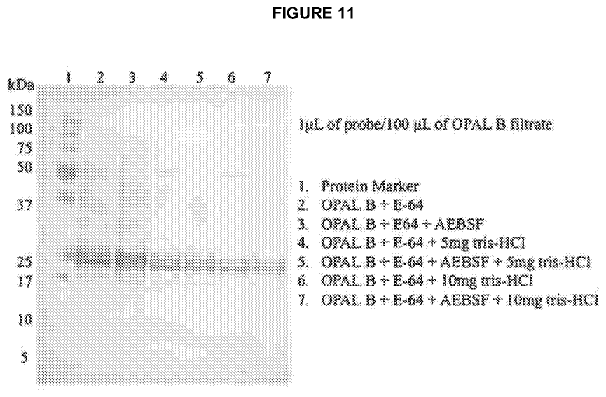

To establish which of the different bands corresponded to a proteolytically active form of subtilase in the OPAL B filtrate, we used a reactivity probe, which specifically labels active serine proteases via a fluorosulfonate group linked to biotin. This reactivity probe can then be visualized on a regular SDS PAGE gel via western blotting technique, using streptavidin-conjugated enzymes such as alkaline phosphatase. Using this technique allowed the visualization of the active serine protease band inhibited by AEBSF treatment at around 50 kDa as shown in FIG. 11. While this band corresponds to one of the observed bands from the chromatography experiments identified as papaya subtilase via mass spectrometry, its molecular weight does not match the published value of either the full-length protein or pro enzyme (70 and 85 kDa respectively). Without wishing to be bound by theory, the fact that we observed several bands originating from the same protein suggests there may be different isoforms of which only the 50 kDa form is proteolytically active.

Overall Conclusions

We have previously described in WO 2004/008887 a process for producing a composition known as OPAL A from the flesh of ripe papaya (Carica papaya). This process involves a heating step followed by treatment with sodium bicarbonate and then a filtration step. Our previous analysis of OPAL A identified cysteine protease activity. OPAL A has shown activity in treating a range of disorders. We have modified the OPAL A process by omitting the heating step to produce OPAL B. We have surprisingly and unexpectedly found that OPAL B contains additional protease activity that is not related to cysteine proteases. We have characterized this as being due to the presence of at least one serine protease which is not present in OPAL A. This protein appears to be present in more than one form, but only a protein having an average molecular weight of 50 kDa as determined by SDS-PAGE showed serine protease activity. LC-MS/MS analysis showed the proteins as having sequences identical to the sequence of a C. papaya sequence putatively identified as a subtilase (Othman and Nuraziyan, 2010), but this was not confirmed by biochemical characterization. Further, this subtilase when expressed recombinantly has a molecular weight of about 70 kDa compared to the 50 kDa molecular weight of the active serine protease that we have presently identified.

Accordingly, we have shown that OPAL B contains a mixture of both one or more proteolytically active serine proteases and one or more proteolytically active cysteine proteases, whereas until now the only known active proteases that have been recognised in Carica papaya have been cysteine proteases.

### Example 6—Quantifying Debriding Potency of OPAL A and OPAL B Filtrate In Vitro

Debridement is the removal of necrotic (dead) tissue and foreign material from wounds to expose underlying viable tissue. This process promotes and accelerates wound healing. We investigated whether the cysteine proteases identified in the OPAL A filtrates would be active in debridement measured using an Artificial Wound Eschar (AWE) debriding assay.

The AWE debriding assay is an in vitro surrogate of wound necrotic tissue proteolysis activity developed by Health Point (Shi, Ermis et al. 2009). It has been shown to compare well to in vivo animal data and was therefore used to assess debriding efficacy of OPAL A filtrate formulations. The AWE substrate consists of a pellet of three wound-related extra cellular matrix proteins, collagen, elastin and fibrin, each tagged with a different fluorophore. Gradual degradation of this matrix can be measured by progressive increase in fluorescence intensity in a Franz diffusion cell setup. The final readings for the experiments are taken at 24 hours.

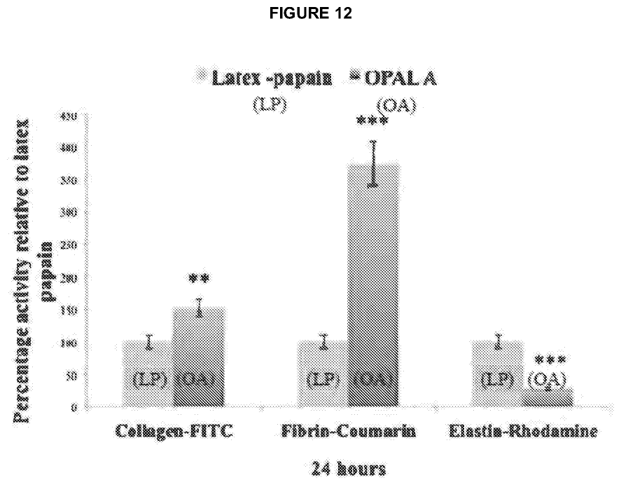

In the first set of experiments, the proteolytic efficacies of native OPAL A filtrate (0.7 mg/mL protein equivalent) was compared against the control crude papain from papaya latex (at 10 mg/mL, Sigma), using a Franz cell diffusion system. The results shown in FIG. 12 clearly indicate that the OPAL A filtrate sample digestion of collagen and fibrin is consistently better than latex papain (10 mg/mL), but weaker with respect to elastin.

### Example 7—Concentrating OPAL a Filtrate Activity Using Dialysis to Improve Debriding Efficacy

The L-BApNA activity of freshly prepared OPAL A filtrate is on average 0.60 IU/mL. The protein content of fresh OPAL A filtrate measures around 0.7 mg/mL, corresponding to a specific solid activity of around 1 IU/mg. Compared to the activity of pure latex papain of around 10 IU/mg, this approximates to about 9% papain content of the OPAL solids. Remarkably the AWE debriding assay performed in Example 6, as shown in FIG. 12, reveals a significantly higher activity of OPAL A than would be expected from its nominal papain content (0.6 IU/mL versus 30 IU/mL for latex papain).

The solid residue content of OPAL A was measured via freeze-drying the extract and yields a consistent value across batches of 0.216±0.005 g/mL. It is therefore clear that the vast majority of OPAL A filtrate solids are potentially non-proteolytic in nature. It may therefore be possible to increase specific activity of the extract by removing the solids from the extracts. To investigate this, we subjected fresh OPAL A filtrate to dialysis using membranes with cut-off sizes of 12 kDa and 25 kDa. Dialysis was carried out for 24 hours at 4° C., after which residual proteolytic activity of the solution was measured via the L-BApNA assay. An aliquot of the solution was also freeze-dried to measure solid content. The treatment caused a small (20%) osmotic volume increase, which was taken into account in the calculations.

Results in Table 5 show almost full retention of activity for either dialysis using cut-off sizes of 12 and 25 kDa and combined with a solid reduction of around 86%.

TABLE 5
Specific activity and solid content of native OPAL A filtrate and dialyzed
OPAL A filtrates with 12 kDa and 25 kDa cut-off molecular sizes.

| | Activity | Volume | Specific activity | Solid content |
|---|---|---|---|---|
| Sample | (IU) | (mL) | (U/mL) | mg/mL |
| OPAL A | 0.216 | 0.166 | 1.301 | 206 |
| Dialyzed | 6.218 | 0.166 | 1.314 | 31 |
| OPAL A | | | | |
| 12 kDa | | | | |
| Dialyzed | 0.205 | 0.166 | 1.232 | 32 |
| OPAL A | | | | |
| 25 kDa | | | | |

This result is consistent with the commonly accepted sizes of Carica papaya proteolytic enzymes (23-30 kDa) and the fact that OPAL A filtrate contains large amounts of low molecular weight sugars, which are removed upon dialysis. The fact that solid content does not significantly change from 12 kDa to 25 kDa dialysis indicates the absence in the composition of molecules with sizes intermediate between these two values. We therefore used the 12 kDa cut-off dialysis membranes for further experiments.

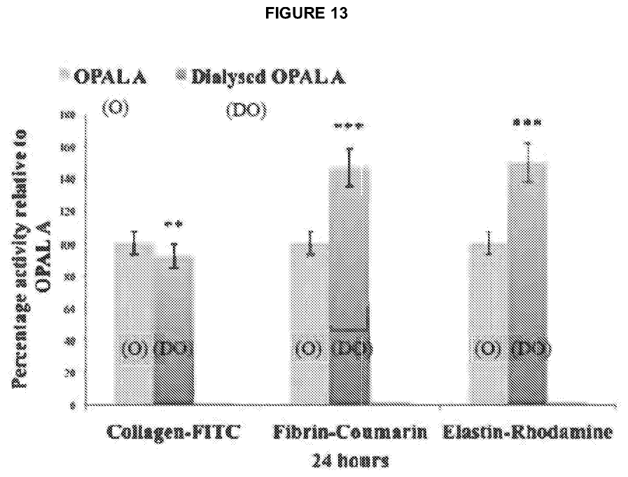

Debriding strength of the OPAL A filtrate versus native Opal A was measured as per Example 6. The data in FIG. 13 show that dialyzed OPAL A is better than native OPAL A at digesting fibrin and elastin but inferior on collagen.

In summary, dialyzed OPAL A filtrate showed full retention of proteolytic activity in comparison with the native OPAL A filtrate, but a solid reduction of 86% less than control. This establishes that the majority of solids in OPAL A filtrate are non-proteolytic in nature, and exist as low molecular weight sugars that can be removed through dialysis. Dialyzed OPAL A filtrate had enhanced debriding activity on both fibrin and elastin as compared to latex papain and native OPAL A, but a marginally minor effect on collagen compared to native OPAL A.

### Example 8—In Vivo Case Study Regarding Management of Severe Pressure Ulcer with OPAL B

This case study discloses rapid treatment of a severe ulcer involving tissue necrosis in an elderly patient through the topical application of OPAL B. OPAL B therefore appears to provide a therapeutic benefit in the healing of severe ulcers through a multifactorial mode of action involving at least tissue regenerative and anti-necrotic/debriding factors.

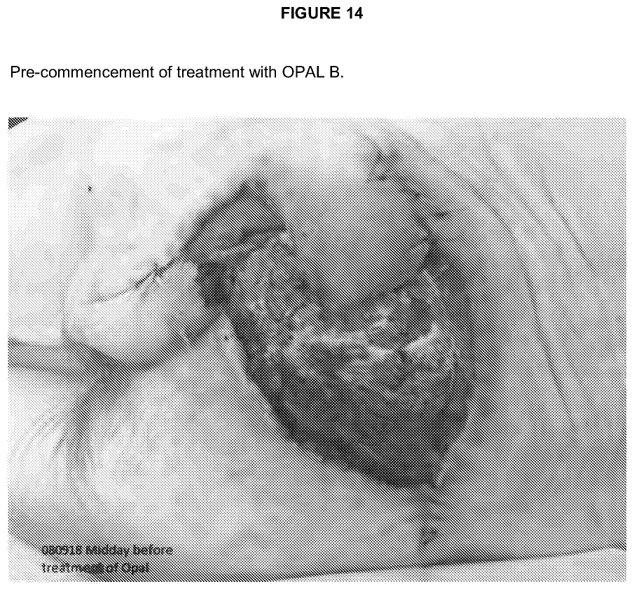

A bed-ridden nursing home patient in her 70s with dementia had developed severe pressure ulcers. The ulcers extended laterally across the majority of the patient's lower back and showed significant tissue necrosis. Upon transfer to a hospital, a vacuum dressing was applied in an attempt to clear excessive exudate produced by the ulcerated wound. Additional surgical intervention to reduce the size of the ulcer with a rhomboid flap was also attempted. Despite continued treatment efforts by hospital staff, the severity of the wound persisted (FIG. 14).

With the consent of the patient's treating surgeon, the vacuum dressing treatment and other surgical interventions were postponed in favour of daily application of fresh, undiluted OPAL B directly to the wound surface, together with daily application of a 30% diluted OPAL B aqueous cream to the skin immediately surrounding the wound.

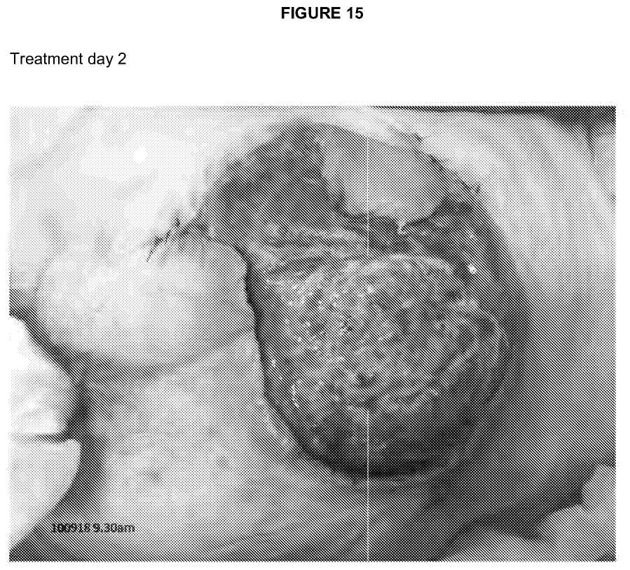

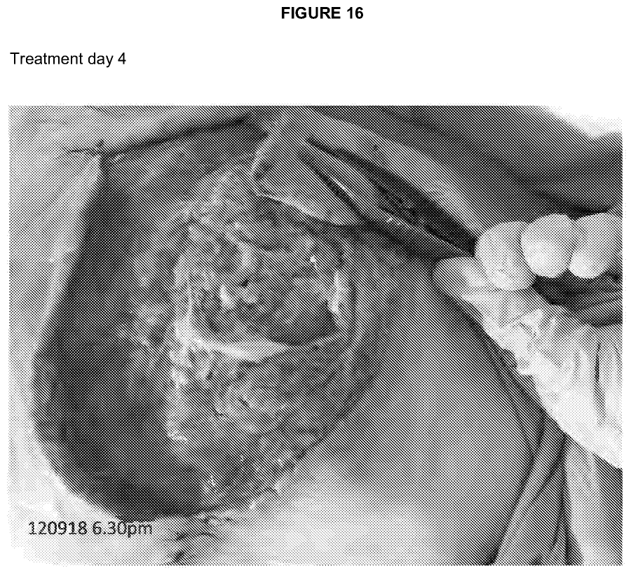

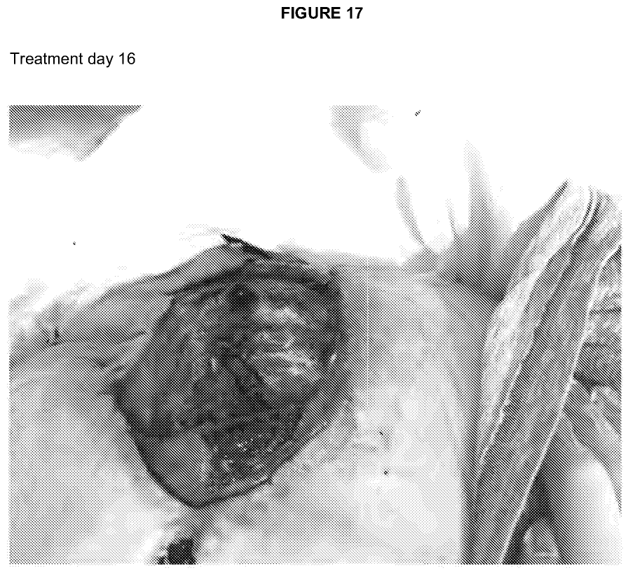

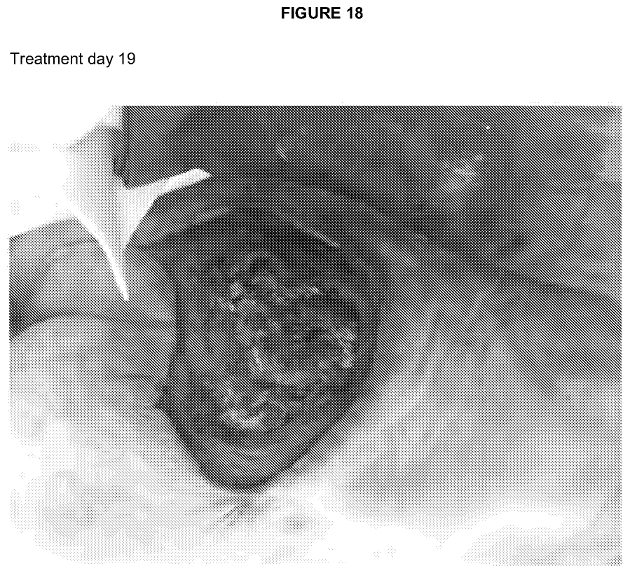

By day 2-4 of Opal B treatment, the wound appeared cleaner and less inflamed (FIGS. 15 and 16). Although some necrotic tissue was still observed, it appeared to be localised to a smaller area of the wound. By days 16-19, further improvement involving reduction in wound size was observed (FIGS. 17 and 18). Upon stabilisation, the wound was surgically debrided. The extremely fast arrest in deterioration/necrosis and the progress made in treating what had been hitherto an extremely severe and untreatable ulcer is surprising and remarkable.

Without wishing to be bound by theory, it is believed that OPAL B may have a number of modes of action: OPAL B appeared to localise tissue that was not viable, and to reverse marginal ischaemia, thereby reducing the amount of necrotic tissue. There is also evidence that OPAL B had begun to debride the slough in the ulcer.

The various features and embodiments of the present invention, referred to in individual sections above apply, as appropriate, to other sections, nutatis mutandis. Consequently, features specified in one section may be combined with features specified in other sections, as appropriate.

All publications mentioned in the above specification are herein incorporated by reference. All of the compositions and/or methods disclosed and claimed in this specification can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention.

### Example 9—Proteome Mapping of OPAL B

In Proteome Mapping of OPAL B using Protein Pilot™ searching the UniProt Viridplantae database, the peptide fragments that were detected are those shown in bold below:

SEQ ID NO: 1 (catalytic triad, Asn 336 and peptides of SEQ ID NOs: 2 to 5 shown in bold)

| | |
|---|---|
| MAVSNPTLYL LSFLLFSISL TPVIASKSSY VVYLGAHSHG LELSSADLDR | 50 |
| VKESHYDFLG SFLGSPEEAQ ESIFYSYTKH INGFAAELND EVAAKLAKHP | 100 |
| KVVSVFLNKG RKLHTTRSWD FLGLEQNGVV PSSSIWKKAR FGEDTIIGNL | 150 |
| DTGVWPESKS FSDEGLGPIP SKWRGICDHG KDSSFHCNRK LIGARFFNRG | 200 |
| YASAVGSLNS SFESPRDNEG HGTHTLSTAG GNMVANASVF GLGKGTAKGG | 250 |
| SPRARVAAYK VCWPPVLGNE CFDADILAAF DAAIHDRVDV LSVSLGGTAG | 300 |
| GFFNDSVAIG SFHAVKHGIV VVCSAGNSGP DDGSVSNVAP WQITVGASTM | 350 |
| DREFPSYVLL GNNMSFKGES LSDAVLPGTN FFPLISALNA KATNASNEEA | 400 |
| ILCEAGALDP KKVKGKILVC LRGLNARVDK GQQAALAGAV GMILANSELN | 450 |
| GNEIIADAHV LPASHISFTD GLSVFEYINL TNSPVAYMTR PKTKLPTKPA | 500 |
| PVMAAFSSKG PNIVTPEILK PDITAPGVNV IAAYTRAQGP TNQNFDRRRV | 550 |
| QFNSVSGTSM SCPHVSGIVG LLKTLYPSWS PAAIRSAIMT SATTMDNINE | 600 |
| SILNASNVKA TPFSYGAGHV QPNQAMNPGL VYDLNTKDYL KFLCALGYSK | 650 |
| TLISIFSNDK FNCPRTNISL ADFNYPSITV PELKGLITLS RKVKNVGSPT | 700 |
| TYRVTVQKPK GISVTVKPKI LKFKKAGEEK SFTVTLKMKA KNPTKEYVFG | 750 |
| ELVWSDEDEH YVRSPIVVKA A | 771 |

Amino acids 1-25 are a putative signal sequence and amino acids 26 to 112 are a putative pro region that is cleaved to form the mature protein.

SEQ ID NO: 6
SWDFLGLEQN GVVPSSSIWK
SEQ ID NO: 7
FGEDTIIGNL DTGVWPESKS FSDEGLGPIP SK
SEQ ID NO: 8
GICDHGKDSS FHCNR
SEQ ID NO: 9
GARFFNRGYA SAVGSLNSSF ESPR
SEQ ID NO: 10
VCWPPVLGNE CFDADILAAF DAAIHDR
SEQ ID NO: 11
HGIVVVCSAG NSGPDDGSVS NVAPWQITVG ASTMDR
SEQ ID NO: 12
FKGESLSDAV LPGTNFFPLI SALNAKATNA SNEEAILCEA GALDPK
SEQ ID NO: 13
ILVCLR
SEQ ID NO: 14
TLPTKPAPVM AAFSSKGPNI VTPEILKPDIT APGVNVIAA
YTRAQGPTNQ NFDR
SEQ ID NO: 15
VQFNSVSGTS MSCPHVSGIV GLLKTLYPSWS PAAIR
SEQ ID NO: 16
ATPFSYGAGH VQPNQAMNPG LVYDLNTK
SEQ ID NO: 17
TLISIFSNDK FNCPRTNISL ADFNYPSITV PELK
SEQ ID NO: 18
GISVTVKPK
SEQ ID NO: 19
EYVFGELVWS DEDEHYVR

### REFERENCES

Arkin A P, Youvan D C. An algorithm for protein engineering: simulations of recursive ensemble mutagenesis, Proc Natl Acad Sci USA. 1992 Aug. 15; 89(16):7811-5.
Delagrave S, Goldman E R, Youvan D C. Recursive ensemble mutagenesis. Protein Eng. 1993 April; 6(3):327-31
Hosseininaveh V, Bandani A, Hosseininaveh F. Digestive proteolytic activity in the Sunn pest, Eurygaster integriceps. J Insect Sci. 2009; 9:1-11.
Kunkel T A. Rapid and efficient site-specific mutagenesis without phenotypic selection. Proc Natl Acad Sci USA. 1985 January; 82(2):488-92.
Kunkel T A, Roberts J D, Zakour R A. Rapid and efficient site-specific mutagenesis without phenotypic selection. Methods Enzymol. 1987; 154:367-82.
Othman and Nuraziyan, Fruit-specific expression of papaya subtilase gene. J. Plant Physiology 2010; 167(2):131-137.
Prescott, Ed., Methods in Cell Biology, Volume XIV, Academic Press, New York, N.Y. (1976)
Rosenberg A H, Goldman E, Dunn J J, Studier F W, Zubay G. Effects of consecutive AGG codons on translation in Escherichia coli, demonstrated with a versatile codon test system. J Bacteriol. 1993 February; 175(3):716-22.
Shi L, Ermis R, Lam K, Cowart J, Attar P, Aust D. Study on the debridement efficacy of formulated enzymatic wound debriding agents by in vitro assessment using artificial wound eschar and by an in vivo pig model. Wound Repair Regen. 2009 November-December; 17(6):853-62.
van der Plas M J, Andersen A S, Nazir S, van Tilburg N H, Oestergaard P R, Krogfelt K A, van Dissel J T, Hensbergen P J, Bertina R M, Nibbering P H. A novel serine protease secreted by medicinal maggots enhances plasminogen activator-induced fibrinolysis. PLoS One. 2014 Mar. 19; 9(3):e92096. Erratum in: PLoS One. 2014; 9(6).
Vinokurov K S, Oppert B, Elpidina E N. An overlay technique for postelectrophoretic analysis of proteinase spectra in complex mixtures using p-nitroanilide substrates. Anal Biochem. 2005 Feb. 1; 337(1):164-6.

1. A composition comprising one or more proteolytically active serine proteases extracted from ripe fruit of Carica papaya.

2. A composition comprising one or more isolated proteolytically active serine proteases extracted from ripe fruit of Carica papaya.

3. A composition comprising a mixture of one or more proteolytically active serine proteases and one or more active cysteine proteases extracted from ripe fruit of Carica papaya.

4. A composition comprising a mixture of one or more isolated proteolytically active serine proteases and one or more active cysteine proteases extracted from ripe fruit of Carica papaya.

5. A process for preparing a composition comprising a proteolytically active serine protease, wherein the process comprises treating pulped ripe fruit of Carica papaya with an alkali, without subjecting the pulped ripe fruit to a heating step, and separating soluble protease from insoluble plant material after the alkali treatment.

6. The process according to claim 5, wherein the alkali is a weak alkali.

7. The process according to claim 6, wherein the weak alkali has a pKa of less than 11.

8. The process according to claim 7, wherein the weak alkali is sodium bicarbonate.

9. The process according to claim 5, wherein the composition further comprises a proteolytically active cysteine protease.

10. The process according to claim 5, wherein the composition is further treated to increase the concentration of the protease or proteases.

11. A composition comprising a proteolytically active serine protease obtainable by the process of claim 5.

12. A composition comprising a proteolytically active serine protease obtained by the process of claim 5.

13. A pharmaceutical composition comprising a proteolytically active Carica papaya serine protease together with a pharmaceutically acceptable carrier or diluent, wherein the serine protease is extracted from ripe fruit of Carica papaya.

14. A pharmaceutical composition comprising an isolated proteolytically active Carica papaya serine protease together with a pharmaceutically acceptable carrier or diluent, wherein the serine protease is extracted from ripe fruit of Carica papaya.

15. A pharmaceutical composition comprising a composition according to claim 1, together with a pharmaceutically acceptable carrier or diluent.

16. A cosmetic composition comprising a proteolytically active Carica papaya serine protease together with a cosmetically acceptable carrier or diluent, wherein the serine protease is extracted from ripe fruit of Carica papaya.

17. A cosmetic composition comprising an isolated proteolytically active Carica papaya serine protease together with a cosmetically acceptable carrier or diluent, wherein the serine protease is extracted from ripe fruit of Carica papaya.

18. A cosmetic composition comprising a composition according to claim 1, together with a cosmetically acceptable carrier or diluent.

19. A pharmaceutical composition according to claim 13 for use in wound debridement.

20. Use of the pharmaceutical composition according to claim 13 in the manufacture of a medicament for wound debridement.

21. A cosmetic composition according to claim 16 for use in skin exfoliation.

22. Use of the cosmetic composition of claim 16 in skin exfoliation.

23. A method of preventing, treating, reducing or ameliorating a skin condition, wherein the method comprises administering to a subject the composition of claim 1.

24. A kit for use in, or when used for, preventing, treating, reducing or ameliorating a skin condition, wherein the kit comprises the composition of claim 1, and instructions for use.


SEQUENCE LISTING (5 sequences)

SEQ ID NO: 1 - protein, 771 aa, Carica papya
MAVSNPTLYLLSFLLFSISLTPVIASKSSYVVYLGAHSHGLELSSADLDRVKESHYDFLGSFLGSPEEAQESIFYSYTKHINGFAAELNDEVAAKLAKHPKVVSVFLNKGRKLHTTRSWDFLGLEQNGVVPSSSIWKKARFGEDTIIGNLDTGVWPESKSFSDEGLGPIPSKWRGICDHGKDSSFHCNRKLIGARFFNRGYASAVGSLNSSFESPRDNEGHGTHTLSTAGGNMVANASVFGLGKGTAKGGSPRARVAAYKVCWPPVLGNECFDADILAAFDAAIHDRVDVLSVSLGGTAGGFFNDSVAIGSFHAVKHGIVVVCSAGNSGPDDGSVSNVAPWQITVGASTMDREFPSYVLLGNNMSFKGESLSDAVLPGTNFFPLISALNAKATNASNEEAILCEAGALDPKKVKGKILVCLRGLNARVDKGQQAALAGAVGMILANSELNGNEIIADAHVLPASHISFTDGLSVFEYINLTNSPVAYMTRPKTKLPTKPAPVMAAFSSKGPNIVTPEILKPDITAPGVNVIAAYTRAQGPTNQNFDRRRVQFNSVSGTSMSCPHVSGIVGLLKTLYPSWSPAAIRSAIMTSATTMDNINESILNASNVKATPFSYGAGHVQPNQAMNPGLVYDLNTKDYLKFLCALGYSKTLISIFSNDKFNCPRTNISLADFNYPSITVPELKGLITLSRKVKNVGSPTTYRVTVQKPKGISVTVKPKILKFKKAGEEKSFTVTLKMKAKNPTKEYVFGELVWSDEDEHYVRSPIVVKAA

SEQ ID NO: 2 - protein, 13 aa, Carica papaya
SFSDEGLGPIPSK

SEQ ID NO: 3 - protein, 24 aa, Carica papaya
GESLSDAVLPGTNFFPLISALNAK

SEQ ID NO: 4 - protein, 27 aa, Carica papaya
GPNIVTPEILKPDITAPGVNVIAAYTR

SEQ ID NO: 5 - protein, 12 aa, Carica papaya
TLYPSWSPAAIR